(19) United States
(12) Patent Application Publication
(10) Pub. No.: US 2025/0283008 A1
(43) Pub. Date: Sep. 11, 2025

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventor: Yutaka TANJI, Tokyo (JP)

(73) Assignee: Resonac Corporation, Tokyo (JP)

(21) Appl. No.: 18/861,283

(22) PCT Filed: Feb. 20, 2024
(86) PCT No.: PCT/JP2024/005960

§ 371 Date: Oct. 29, 2024

(30) Foreign Application Priority Data
Feb. 20, 2023 (JP) 2023-024274

(51) Int. Cl. C10M 105/70; C07D 215/227; C07D 311/18; C07D 311/80; C10M 105/54; C10N 40/18; G11B 5/725

(52) U.S. Cl. CPC C10M 105/70; C07D 215/227; C07D 311/18; C07D 311/80; C10M 105/54; G11B 5/7257; C10M 2211/0445; C10M 2215/221; C10N 2040/18

(57) ABSTRACT

Provided is a fluorine-containing ether compound represented by the following formula. R¹—R²—CH₂—R³[—CH₂—R⁴—CH₂—R³]ₙ—CH₂—R⁵—R⁶ (n represents an integer of 0 to 2, R³ represents a perfluoropolyether chain, R⁴, R², and R⁵ represent a divalent linking group having one or more polar groups, R² and R⁵ have an oxygen atom at an end bonded to R¹ or R⁶, R¹ and R⁶ represent an organic group having 1 to 50 carbon atoms or a hydrogen atom, at least one of R¹ or R⁶ is represented by Formula (2), one of X and Y represents C═O, and the other represents —NH—, —NR⁷— (R⁷ represents a hydrocarbon group having 1 to 8 carbon atoms), or —O—, Q represents a 5- to 8-membered heterocyclic ring structure, which is formed of a carbon atom of a linking group indicated by a dashed arc together with X and Y, and Z represents 0 to 5 substituents.)

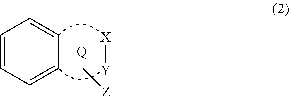

### TECHNICAL FIELD

The present invention relates to a fluorine-containing ether compound, a lubricant for a magnetic recording medium, and a magnetic recording medium.

Priority is claimed on Japanese Patent Application No. 2023-024274, filed Feb. 20, 2023, the content of which is incorporated herein by reference.

### BACKGROUND ART

In order to improve the recording density of a magnetic recording and reproducing device, development of a magnetic recording medium suitable for a high recording density has been promoted.

In the related art, as a magnetic recording medium, there is a magnetic recording medium in which a recording layer is formed on a substrate and a protective layer made of carbon or the like is formed on the recording layer. The protective layer protects information recorded on the recording layer and increases the slidability of a magnetic head. However, the durability of the magnetic recording medium is not sufficiently obtained only by providing the protective layer on the recording layer. Therefore, in general, a lubricating layer is formed by coating a surface of the protective layer with a lubricant.

As a lubricant used in a case of forming a lubricating layer of a magnetic recording medium, for example, a lubricant which contains a compound containing a polar group such as a hydroxy group or an amino group at an end of a fluorine-based polymer having a repeating structure containing —CF₂— has been proposed (see, for example, Patent Documents 1 to 5).

Patent Document 1 discloses a fluorine-containing ether compound in which divalent linking groups having a polar group are each linked to both ends of a perfluoropolyether chain, and end groups having a heterocyclic ring are each bonded to the both sides thereof.

Patent Document 2 discloses a fluorine-containing ether compound in which divalent linking groups having a polar group are each linked to both ends of a perfluoropolyether chain, and an end group substituted with an organic group having 1 to 8 carbon atom and at least one hydrogen that has an amide bond is bonded to at least one of the both sides thereof.

Patent Documents 3 and 4 disclose a fluorine-containing ether compound in which divalent linking groups having a polar group are each linked to both ends of a perfluoropolyether chain, and an end group having an aromatic ring is bonded to the one or both sides thereof.

Patent Document 5 discloses a compound having a skeleton in which perfluoropolyethers are each bonded to both sides of an aliphatic hydrocarbon chain having a hydroxy group present in the central portion of a molecule, and an aromatic ring may be present at the end thereof.

### CITATION LIST

### Patent Documents

Patent Document 1: PCT International Publication No. WO2018/139174
Patent Document 2: PCT International Publication No. WO2019/039265
Patent Document 3: Japanese Patent Publication No. 5909837
Patent Document 4: Japanese Patent Publication No. 6040455
Patent Document 5: Japanese Patent Publication No. 6804981

### SUMMARY OF INVENTION

### Technical Problem

In the magnetic recording and reproducing device, it is required to further reduce the floating amount of the magnetic head. Therefore, it is required to further reduce the thickness of the lubricating layer in the magnetic recording medium.

However, in general, in a case where the thickness of the lubricating layer is reduced, the cotability of the lubricating layer are degraded, and the chemical substance resistance of the magnetic recording medium tends to be degraded. In addition, in a case where the thickness of the lubricating layer is reduced, the adhesion between the lubricating layer, which covers the surface of the protective layer, and the protective layer is insufficient, and a pickup in which the fluorine-containing ether compound in the lubricating layer adheres to the magnetic head is likely to occur.

The present invention has been made in consideration of the above-described circumstances, and an object thereof is to provide a fluorine-containing ether compound which is capable of forming a lubricating layer with excellent chemical substance resistance even in a case where the thickness thereof is small and with suppressed pickup and which is suitably used as a material of a lubricant for a magnetic recording medium.

Another object of the present invention is to provide a lubricant for a magnetic recording medium, which contains the fluorine-containing ether compound of the present invention.

Still another object of the present invention is to provide a magnetic recording medium which is provided with a lubricating layer containing the fluorine-containing ether compound of the present invention and has excellent reliability and durability.

### Solution to Problem

The present invention includes the following aspects.

[1]A fluorine-containing ether compound which is represented by Formula (1).

R¹—R²—CH₂—R³[—CH₂—R⁴—CH₂—R³]—CH₂—R⁵—R⁶ (1)

(In Formula (1), n represents an integer of 0 to 2. R³ represents a perfluoropolyether chain. (n+1) pieces of R³'s may be partially or entirely the same as or different from each other. R⁴ represents a divalent linking group having one or more polar groups. Two R⁴'s may be the same as or different from each other in a case where n represents 2. Each R² and R⁵ represents a divalent linking group having one or more polar groups. R² and R⁵ may be the same as or different from each other. R² has an oxygen atom at an end bonded to R¹. R⁵ has an oxygen atom at an end bonded to R⁶. Each R¹ and R⁶ represents an organic group having 1 to 50 carbon atoms, which is bonded to an oxygen atom included in R² or R⁵, or a hydrogen atom. R¹ and R⁶ may be the same as or different from each other. At least one of R¹ or R⁶ has a structure represented by Formula (2).)

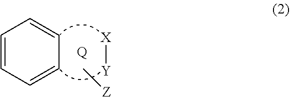

(In Formula (2), one of X and Y represents a carbonyl group (C═O), and the other represents —NH—, —NR⁷— (R⁷ represents a hydrocarbon group having 1 to 8 carbon atoms), or —O—. Q represents that a carbon atom of a linking group indicated by a dashed arc forms a 5- to 8-membered heterocyclic ring structure together with X and Y Z represents 0 to 5 substituents. Z's each independently represent an organic group having 1 to 8 carbon atoms. Z may be bonded to any of carbon atoms constituting two rings in Formula (2) (where a carbonyl carbon atom of X or Y is excluded). In a case where two or more Z's are present, two Z's may be bonded to each other to form a ring structure. Any of the carbon atoms constituting the two rings in Formula (2) (where a carbonyl carbon atom of X or Y is excluded) is bonded to the oxygen atom at the end of R² or R⁵.)

[2] The fluorine-containing ether compound according to [1], in which Formula (2) is a structure represented by Formula (2-1).

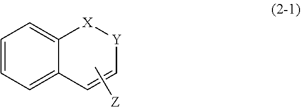

(In Formula (2-1), one of X and Y represents a carbonyl group (C═O), and the other represents —NH—, —NR⁷— (R⁷ represents a hydrocarbon group having 1 to 8 carbon atoms), or —O—. Z represents 0 to 5 substituents. Z's each independently represent an organic group having 1 to 8 carbon atoms. Z may be bonded to any of carbon atoms constituting two rings in Formula (2-1) (where a carbonyl carbon atom of X or Y is excluded). In a case where two or more Z's are present, two Z's may be bonded to each other to form a ring structure. Any of the carbon atoms constituting the two rings in Formula (2-1) (where a carbonyl carbon atom of X or Y is excluded) is bonded to the oxygen atom at the end of R² or R⁵.)

[3] The fluorine-containing ether compound according to [1] or [2], in which R¹ and R⁶ in Formula (1) are the same as each other.

[4] The fluorine-containing ether compound according to [1] or [2], in which one of R¹ and R⁶ in Formula (1) represents the structure represented by Formula (2) and the other represents an organic group, an alkyl group, or a hydrogen atom, the organic group having at least one double bond or triple bond and being other than the structure represented by Formula (2), the alkyl group having 1 to 8 carbon atoms, which may have a substituent.

[5] The fluorine-containing ether compound according to any one of [1] to [4], in which the organic group as Z in Formula (2) is selected from the group consisting of an alkyl group, an alkenyl group, an alkynyl group, an aryl group, an alkyl group substituted with an aryl group, and an alkoxy group.

[6] The fluorine-containing ether compound according to any one of [1] to [5], in which all the polar groups in R², R⁴, and R⁵ in Formula (1) are hydroxy groups.

[7] The fluorine-containing ether compound according to any one of [1] to [5], in which some or all of the polar groups in R², R⁴, and R⁵ in Formula (1) are hydroxy groups, and a total number of hydroxy groups included in R², hydroxy groups included in R⁴, and hydroxy groups included in R⁵ is in a range of 2 to 8.

[8] The fluorine-containing ether compound according to any one of [1] to [7], in which R² and R⁵ in Formula (1) each independently represent a linking group represented by Formula (3).

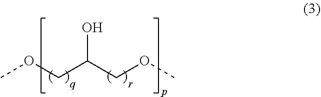

(In Formula (3), p represents an integer of 1 to 3. q represents an integer of 1 to 5. r represents an integer of 1 to 5. In a case where p represents 2 or greater, combinations of q and r in each structural unit may be the same as or different from each other. In Formula (3), an oxygen atom at an end on a left side is bonded to a group disposed on an R¹ side of R² or R⁵ in Formula (1). In Formula (3), an oxygen atom at an end on a right side is bonded to a group disposed on an R⁶ side of R² or R⁵ in Formula (1)).

[9] The fluorine-containing ether compound according to any one of [1] to [8], in which R² in Formula (1) represents a linking group represented by Formula (3-1) or (3-2), and R⁵ in Formula (1) represents a linking group represented by Formula (3-3) or (3-4).

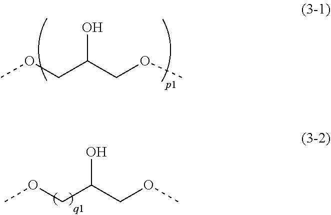

(p1 in Formula (3-1) represents an integer of 1 to 3. q1 in Formula (3-2) represents an integer of 2 to 4. In Formulae (3-1) and (3-2), an oxygen atom at an end on a left side is bonded to R¹ in Formula (1).)

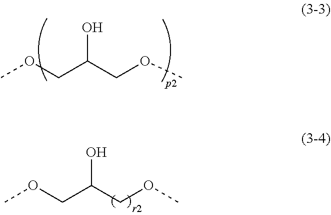

(p2 in Formula (3-3) represents an integer of 1 to 3. r2 in Formula (3-4) represents an integer of 2 to 4. In Formulae (3-3) and (3-4), an oxygen atom at an end on a right side is bonded to R⁶ in Formula (1).)

[10] The fluorine-containing ether compound according to any one of [1] to [9], in which in Formula (1), the (n+1) pieces of R³'s are the same as each other, and R¹—R²— and R⁶—R⁵— are the same as each other.

[11] The fluorine-containing ether compound according to any one of [1] to [10], in which n pieces of R⁴'s in Formula (1) each independently represent a linking group represented by Formula (4).

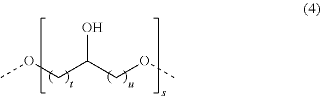

(In Formula (4), s represents an integer of 1 to 3. t represents an integer of 1 to 5. u represents an integer of 1 to 5. In a case where s represents 2 or greater, combinations of t and u in each structural unit may be the same as or different from each other. In Formula (4), an oxygen atom at an end on a left side is bonded to a group disposed on an R¹ side of R⁴ in Formula (1). In Formula (4), an oxygen atom at an end on a right side is bonded to a group disposed on an R⁶ side of R⁴ in Formula (1).)

[12] The fluorine-containing ether compound according to any one of [1] to [11] in which n pieces of R⁴'s in Formula (1) each independently represent a linking group represented by any of Formulae (4-1) to (4-3).

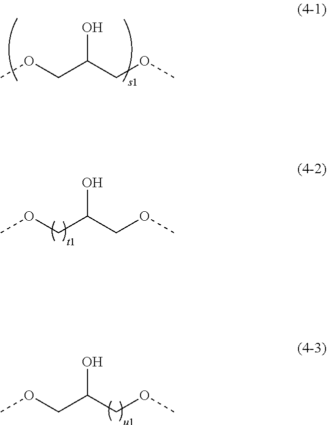

(s1 in Formula (4-1) represents an integer of 1 to 3.)

(t1 in Formula (4-2) represents an integer of 2 to 4. In Formula (4-2), an oxygen atom at an end on a left side is bonded to a methylene group adjacent to an R¹ side of R⁴ in Formula (1).)

(u1 in Formula (4-3) represents an integer of 2 to 4. In Formula (4-3), an oxygen atom at an end on a right side is bonded to a methylene group adjacent to an R⁶ side of R⁴.)

[13] The fluorine-containing ether compound according to any one of [1] to [12], in which (n+1) pieces of R³'s in Formula (1) each independently represent a perfluoropolyether chain represented by Formula (5).

−(CF₂)_{w1}−O—(CF₂O)_{w2}−(CF₂CF₂O)_{w3}−(CF₂CF₂CF₂O)_{w4}−(CF₂CF₂CF₂CF₂O)_{w5}−(CF₂)_{w6}− (5)

(In Formula (5), w2, w3, w4, and w5 represent an average degree of polymerization, and each independently represent 0 to 20. Here, all of w2, w3, w4, and w5 do not represent 0 at the same time. w1 and w6 represent an average value representing the number of CF₂'s, and each independently represent 1 to 3. The arrangement order of (CF₂O), (CF₂CF₂O), (CF₂CF₂CF₂O), and (CF₂CF₂CF₂CF₂O), which are the repeating units in Formula (5), is not particularly limited.)

[14] The fluorine-containing ether compound according to any one of [1] to [13], in which (n+1) pieces of R³'s in Formula (1) each independently represent any one selected from the group consisting of perfluoropolyether chains each represented by any of Formulae (5-1) to (5-4).

—CF₂—(OCF₂CF₂)ₕ—(OCF₂)ᵢ—OCF₂— (5-1)

(In Formula (5-1), h and i represent an average degree of polymerization, where h represents 1 to 20 and i represents 0 to 20.)

—CF₂CF₂—(OCF₂CF₂CF₂)ⱼ—OCF₂CF₂— (5-2)

(In Formula (5-2), j represents an average degree of polymerization, and represents 1 to 15.)

—CF₂CF₂CF₂—(OCF₂CF₂CF₂CF₂)ₖ—OCF₂CF₂CF₂— (5-3)

(In Formula (5-3), k represents an average degree of polymerization, and represents 1 to 10.)

—(CF₂)_{w7}—O—(CF₂CF₂CF₂O)_{w8}—(CF₂CF₂O)_{w9}—(CF₂)_{w10}— (5-4)

(In Formula (5-4), w8 and w9 represent an average degree of polymerization, and each independently represents 1 to 20. w7 and w10 represent an average value representing the number of CF₂'s, and each independently represent 1 to 2.)

[15] The fluorine-containing ether compound according to any one of [1] to [14], in which the fluorine-containing ether compound has a number-average molecular weight of 500 to 10000.

[16]A lubricant for a magnetic recording medium, including: the fluorine-containing ether compound according to any one of [1] to [15].

[17]A magnetic recording medium, which is provided with at least a magnetic layer, a protective layer, and a lubricating layer in this order on a substrate, in which the lubricating layer contains the fluorine-containing ether compound according to any one of [1] to [15].

[18] The magnetic recording medium according to [17], in which the lubricating layer has an average film thickness of 0.5 nm to 2.0 nm.

### Advantageous Effects of Invention

The fluorine-containing ether compound according to the embodiment of the present invention is a compound represented by Formula (1), and is suitable as a material for a lubricant for a magnetic recording medium.

Since the lubricant for a magnetic recording medium of the present invention contains the fluorine-containing ether compound of the present invention, a lubricating layer which has excellent chemical substance resistance and suppressed pickup can be formed even in a case where the thickness thereof is reduced.

Since the magnetic recording medium of the present invention is provided with a lubricating layer having excellent chemical substance resistance and suppressed pickup, the magnetic recording medium has excellent reliability and durability.

### BRIEF DESCRIPTION OF DRAWINGS

FIG. 1 A schematic cross-sectional view showing an embodiment of a magnetic recording medium according to the present invention.

### DESCRIPTION OF EMBODIMENTS

Hereinafter, a fluorine-containing ether compound, a lubricant for a magnetic recording medium (hereinafter, also referred to as “lubricant”), and a magnetic recording medium of the present invention will be described in detail. Further, the present invention is not limited to the embodiments described below.

Here, the reason why, in a case where a lubricating layer is formed on a protective layer of a magnetic recording medium using a lubricant containing a fluorine-containing ether compound according to the present embodiment, a lubricating layer having excellent chemical substance resistance and suppressed pickup can be formed even in a case where the thickness thereof is small will be described.

As shown in Formula (1), the fluorine-containing ether compound of the present embodiment has a perfluoropolyether chain represented by R³ (hereinafter, also referred to as “PFPE chain”). In a case where a lubricating layer is formed by coating a protective layer with a lubricant containing the fluorine-containing ether compound, the PFPE chain coats the surface of the protective layer and imparts lubricity to the lubricating layer, and thus a frictional force between the magnetic head and the protective layer is reduced.

As shown in Formula (1), end groups represented by R¹ and R⁶ are bonded to both end portions of a skeleton (—CH₂—R³[—CH₂—R⁴—CH₂—R³]ₙ—CH₂—) having a PFPE chain represented by R³ through R² and R⁵ which represent a divalent linking group having one or more polar groups. At least one of R¹ or R⁶ represents a condensed aromatic group represented by Formula (2) in which a benzene ring and a heterocyclic ring having an amide or an ester with high polarity are conjugated and condensed. In a case where the condensed aromatic group represented by Formula (2) has an amide, the condensed aromatic group has a wide planar structure from the benzene ring to the amide, formed of carbon, oxygen, and nitrogen having sp² hybrid orbitals. In addition, in a case where the condensed aromatic group represented by Formula (2) has an ester, the condensed aromatic group has a wide planar structure from the benzene ring to the ester, formed of carbon and oxygen having sp² hybrid orbitals. The wide planar structure of the condensed aromatic group represented by Formula (2) can closely adhere to the protective layer in a plane. As a result, it is presumed that the lubricant containing the fluorine-containing ether compound represented by Formula (1) exhibits an extremely strong interaction with the protective layer, and even in a case where the coating amount is reduced and the thickness is reduced, the surface of the protective layer can be coated at a high coating rate, and thus a lubricating layer having excellent chemical substance resistance can be formed. Therefore, the lubricant containing the fluorine-containing ether compound according to the present embodiment can contribute to a decrease in film thickness (reduction of magnetic spacing) of the lubricating layer.

In addition, since the condensed aromatic group represented by Formula (2) is relatively rigid, free rotation is difficult to carry out. Therefore, the condensed aromatic group represented by Formula (2) in Formula (1) and the polar group in R² and R⁵ are difficult to interact with each other, and the ability to inhibit each interaction with the protective layer is extremely low. Therefore, in the fluorine-containing ether compound represented by Formula (1), the condensed aromatic group represented by Formula (2) and the polar group of R² and R⁵ each independently exhibit a satisfactory interaction with the protective layer, and can be each independently bonded to a plurality of functional groups (active points) present on the protective layer. In this manner, it is presumed that the lubricating layer containing the fluorine-containing ether compound represented by Formula (1) has excellent adhesion to the protective layer, and the fluorine-containing ether compound which is present without adhering to the protective layer is aggregated to prevent the fluorine-containing ether compound from being attached to the magnetic head as foreign matter (smear), and thus the pickup is suppressed.

As described above, it is presumed that the lubricant containing the fluorine-containing ether compound according to the present embodiment can firmly cover the surface of the protective layer at a high coating rate even in a case where the thickness thereof is small, and can form a lubricating layer with excellent chemical substance resistance and suppressed pickup.

Further, the present inventors have confirmed that a lubricating layer having satisfactory chemical substance resistance and suppressed pickup can be formed by using the lubricant containing the fluorine-containing ether compound described above, thereby completing the present invention.

Hereinafter, the fluorine-containing ether compound, the lubricant for a magnetic recording medium, and the magnetic recording medium of the present invention will be described in detail. Further, the present invention is not limited to the embodiments described below.

### [Fluorine-Containing Ether Compound]

The fluorine-containing ether compound of the present embodiment is represented by Formula (1).

R¹—R²—CH₂—R³[—CH₂—R⁴—CH₂—R³]—CH₂—R⁵—R⁶ (1)

(In Formula (1), n represents an integer of 0 to 2. R³ represents a perfluoropolyether chain. (n+1) pieces of R³'s may be partially or entirely the same as or different from each other. R⁴ represents a divalent linking group having one or more polar groups. Two R⁴'s may be the same as or different from each other in a case where n represents 2. Each R² and R⁵ represents a divalent linking group having one or more polar groups. R² and R⁵ may be the same as or different from each other. R² has an oxygen atom at an end bonded to R¹. R⁵ has an oxygen atom at an end bonded to R⁶. Each R¹ and R⁶ represents an organic group having 1 to 50 carbon atoms, which is bonded to an oxygen atom included in R² or R⁵, or a hydrogen atom. R¹ and R⁶ may be the same as or different from each other. At least one of R¹ or R⁶ has a structure represented by Formula (2).)

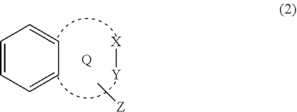

(In Formula (2), one of X and Y represents a carbonyl group (C═O), and the other represents —NH—, —NR⁷— (R⁷ represents a hydrocarbon group having 1 to 8 carbon atoms), or —O—. Q represents that a carbon atom of a linking group indicated by a dashed arc forms a 5- to 8-membered heterocyclic ring structure together with X and Y Z represents 0 to 5 substituents. Z's each independently represent an organic group having 1 to 8 carbon atoms. Z may be bonded to any of carbon atoms constituting two rings in Formula (2) (where a carbonyl carbon atom of X or Y is excluded). In a case where two or more Z's are present, two Z's may be bonded to each other to form a ring structure. Any of the carbon atoms constituting the two rings in Formula (2) (where a carbonyl carbon atom of X or Y is excluded) is bonded to the oxygen atom at the end of R² or R⁵.

The fluorine-containing ether compound of the present embodiment has a structure represented by Formula (1), in which a divalent linking group having one or more polar groups, represented by R² and R⁵, and an end group represented by R¹ and R⁶ are bonded in this order to both sides of a skeleton (—CH₂—R³[—CH₂—R⁴—CH₂—R³]ₙ—CH₂—) having a PFPE chain represented by R³, and at least one end group is represented by Formula (2). n which represents the number of repeating units [—CH₂—R⁴—CH₂—R³] in Formula (1) is 0 to 2. In a case where n is 1 or 2, since the fluorine-containing ether compound has R⁴ having a polar group, in a case where a lubricant containing this compound is used to form a lubricating layer on the protective layer, a suitable interaction occurs between the lubricating layer and the protective layer, and the lubricating layer can be formed to have further suppressed pickup, which is preferable. In addition, since n is 2 or less, a polar group which is not involved in the interaction with the protective layer is unlikely to be generated as compared with a case where n is 3 or greater, and thus a lubricating layer with satisfactory chemical substance resistance can be formed in which contaminants are less likely to be taken in by polar groups that are not involved in the interaction. n can be appropriately determined depending on the applications and the like of the fluorine-containing ether compound.

(Linking Group Represented by R² and R⁵)

Each R² and R⁵ represents a divalent linking group having one or more polar groups. R² has an oxygen atom at an end bonded to R¹, and in a case where R¹ represents an organic group, R² is bonded to R¹ through an ether bond. R⁵ has an oxygen atom at an end bonded to R⁶, and in a case where R⁶ represents an organic group, R⁵ is bonded to R⁶ through an ether bond. In the fluorine-containing ether compound represented by Formula (1), since R² and R⁵ have a polar group, in a case where a lubricant containing the fluorine-containing ether compound is used to form a lubricating layer on the protective layer, a suitable interaction occurs between the lubricating layer and the protective layer. R² and R⁵ can be appropriately selected according to the performance required for the lubricant containing the fluorine-containing ether compound.

Examples of the polar group included in R² and R⁵ include a hydroxy group (—OH), an amino group (—NH₂), a carboxy group (—COOH), a formyl group (—((C═O)H), a carbonyl group (—CO—), and a sulfo group (—SO₃H). The number of polar groups included in R² and R⁵ is preferably 1 to 3 and more preferably 1 or 2. In a case where R² and/or R⁵ has two or more polar groups, the kinds of the polar groups may be partially or entirely the same as or different from each other.

It is preferable that R² and R⁵ each include a hydroxy group as the polar group and more preferable that all the polar groups included in R² and R⁵ are hydroxy groups. The hydroxy group has a large interaction with a protective layer, particularly a protective layer formed of a carbon-based material. Therefore, in a case where R² and/or R⁵ includes a hydroxy group as the polar group, the lubricating layer containing the fluorine-containing ether compound has high adhesion to the protective layer.

In a case where R² and R⁵ each include a hydroxy group as the polar group, the total number of the hydroxy groups included in R² and the hydroxy groups included in R⁵ in Formula (1) is preferably 2 to 6 and more preferably 2 to 4. In a case where the total number of the above-described hydroxy groups is 2 or more, the interaction between the hydroxy groups contained in R² and R⁵ and the protective layer is more effectively obtained. As a result, the fluorine-containing ether compound can form a lubricating layer having higher adhesion to the protective layer. In addition, in a case where the total number of the above-described hydroxy groups is 6 or less, since the hydroxy groups which are not involved in the bonding between the lubricating layer and the active points on the protective layer can prevent the environmental substances which generate the contaminants from being attracted to the lubricating layer, the contamination with the chemical substances is suppressed.

It is preferable that the divalent linking group represented by R² has an oxygen atom at an end bonded to R¹ and also has an oxygen atom disposed at the other end (end bonded to a methylene group (—CH₂—) adjacent to R²) thereof. In addition, it is preferable that the divalent linking group represented by R⁵ has an oxygen atom at an end bonded to R⁶ and also has an oxygen atom disposed at the other end (end bonded to a methylene group adjacent to R⁵). The oxygen atom disposed at an end of the divalent linking group represented by R² and R⁵ forms an ether bond (—O—) with the methylene group adjacent to R² and R⁵. The ether bond imparts moderate flexibility to the fluorine-containing ether compound represented by Formula (1), and increases the affinity between the polar group included in the linking group and the protective layer.

The divalent linking group represented by R² and R⁵ is preferably an alkylene group having oxygen atoms at both ends and 3 to 9 carbon atoms, in which one or two hydrogen atoms are substituted with a hydroxy group, and more preferably an alkylene group having 3 to 5 carbon atoms. The alkylene group may have an ether bond between carbon atoms.

Specifically, it is preferable that R² and R⁵ each independently represent a linking group represented by Formula (3). In a case where each R² and R⁵ represents a linking group represented by Formula (3), R¹—R²— and —R⁵—R⁶ are more flexible, and the interaction with the protective layer is further enhanced.

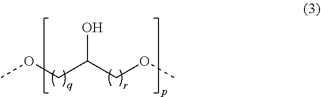

(In Formula (3), p represents an integer of 1 to 3. q represents an integer of 1 to 5. r represents an integer of 1 to 5. In a case where p represents 2 or greater, combinations of q and r in each structural unit may be the same as or different from each other. In Formula (3), the oxygen atom at the end on the left side is bonded to a group disposed on the R¹ side of R² or R⁵ in Formula (1). In Formula (3), an oxygen atom at an end on a right side is bonded to a group disposed on an R⁶ side of R² or R⁵ in Formula (1).

In the linking group represented by Formula (3), p represents an integer of 1 to 3. Since p represents 1 or greater, the linking group represented by R² or R⁵ has 1 or more hydroxy groups, and thus a fluorine-containing ether compound which forms a lubricating layer having satisfactory adhesion to the protective layer is obtained. In addition, since p represents 3 or less, since the number of hydroxy groups in the linking group represented by R² or R⁵ is large, the polarity of the fluorine-containing ether compound is extremely high, and thus occurrence of contamination due to the chemical substance can be prevented.

In the linking group represented by Formula (3), it is preferable that p represents 1 or 2. In a case where p represents 1 or 2, the number of hydroxy groups in the linking group represented by R² or R⁵ is appropriate, contaminants due to the hydroxy group that is not involved in the bonding to the active point on the protective layer are unlikely to be taken into the layer, and thus a lubricating layer with more satisfactory chemical substance resistance can be formed.

In the linking group represented by Formula (3), q and r each represent an integer of 1 to 5. Since q and r each represent an integer of 1 to 5, the number of carbon atoms in the molecule is not extremely increased by the methylene group included in Formula (3). Therefore, the fluorine-containing ether compound represented by Formula (1) is likely to have an appropriate proportion of fluorine atoms in the entire molecule, and thus a lubricating layer having excellent lubricity can be formed. It is preferable that q and r each represent 1 to 4.

In a case where p in Formula (3) represents 2 or greater, combinations of q and r in each structural unit (—(CH₂)_{q}—CH(OH)—(CH₂)ᵣ—O—) may be the same as or different from each other. It is preferable that at least one of q or r in each structural unit represents 1.

It is more preferable that R² represents a linking group represented by Formula (3-1) or (3-2).

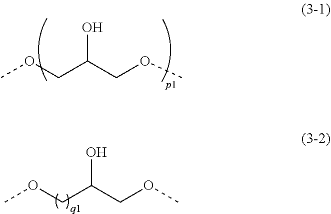

(p1 in Formula (3-1) represents an integer of 1 to 3. q1 in Formula (3-2) represents an integer of 2 to 4. In Formulae (3-1) and (3-2), the oxygen atom at the end on the left side is bonded to R¹ in Formula (1).)

It is more preferable that R⁵ in Formula (1) represents a linking group represented by Formula (3-3) or (3-4).

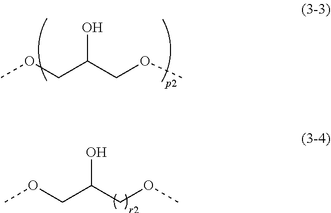

(p2 in Formula (3-3) represents an integer of 1 to 3. r2 in Formula (3-4) represents an integer of 2 to 4. In Formulae (3-3) and (3-4), the oxygen atom at the end on the right side is bonded to R⁶ in Formula (1).)

The linking group represented by Formulae (3-1) to (3-4) is a linking group having a hydroxy group, which has a particularly large interaction with the protective layer, among the polar groups. In addition, in the linking group represented by Formulae (3-1) to (3-4), a methylene group is disposed on both sides of carbon atoms to which a hydroxy group is bonded. Therefore, in a case where R² represents a linking group represented by Formula (3-1) or (3-2) and in a case where R⁵ represents a linking group represented by Formula (3-3) or (3-4), a fluorine-containing ether compound capable of forming a lubricating layer with higher adhesion to the protective layer is obtained due to the reasons <1> and <2> described below.

<1> At least a methylene group and an oxygen atom (—O—) of R² or R⁵ are disposed between the carbon atom to which a hydroxy group contained in the linking group represented by Formulae (3-1) to (3-4) is bonded and R¹ or R⁶. Therefore, the distance between the hydroxy group contained in the linking group represented by Formulae (3-1) to (3-4) and the end group represented by R¹ and/or R⁶ is appropriate. In addition, since the free rotation of the end group is slightly suppressed by the oxygen atom which is a linking portion of R¹ and R² and R⁵ and R⁶, the end group represented by R¹ and/or R⁶ and the hydroxy group of the linking group represented by Formulae (3-1) to (3-4) are unlikely to interact with each other. Therefore, the hydroxy group contained in the linking group represented by Formulae (3-1) to (3-4) and the end group represented by R¹ and/or R⁶ each independently exhibit a satisfactory interaction with the protective layer, and are each independently easily bonded to a plurality of functional groups (active points) present on the protective layer.

<2> In a case where R² represents a linking group represented by Formula (3-1) or (3-2), R² and R¹ are bonded to each other via an oxygen atom, and R² and a methylene group adjacent to R² are bonded to each other via an oxygen atom. In addition, in a case where R⁵ represents a linking group represented by Formula (3-3) or (3-4), R⁵ and R⁶ are bonded to each other via an oxygen atom, and R⁵ and a methylene group adjacent to R⁵ are bonded to each other via an oxygen atom. As a result, the fluorine-containing ether compound has moderate flexibility, and a lubricating layer having more satisfactory chemical substance resistance can be formed.

In the linking group represented by Formulae (3-1) and (3-3), p1 and p2 each represent an integer of 1 to 3. Since p1 and p2 represent 1 or greater, a fluorine-containing ether compound capable of obtaining a lubricating layer with satisfactory adhesion to the protective layer is obtained. In addition, since p1 and p2 represent 3 or less, the number of hydroxy groups in the linking group represented by Formulae (3-1) and (3-3) is not increased, and thus the polarity of the fluorine-containing ether compound is not extremely high so that occurrence of contamination due to the chemical substance can be prevented.

In the linking group represented by Formulae (3-1) and (3-3), in a case where p1 and p2 each represent 2 or 3, the distance between the hydroxy groups contained in the linking group represented by Formulae (3-1) and (3-3) is appropriate. As a result, even in a case where a plurality of hydroxy groups included in R² or R⁵ are present, the hydroxy groups included in R² or R⁵ are each likely to be involved in the bonding to the active points on the protective layer. In the linking group represented by Formulae (3-1) and (3-3), it is preferable that p1 and p2 each represent 1 or 2. It is more preferable that p1 and p2 represent 1 from the viewpoint of obtaining a fluorine-containing ether compound which is easily synthesized.

In the linking group represented by Formulae (3-2) and (3-4), q1 and r2 each represent an integer of 2 to 4. Since q1 and r2 represent an integers of 2 to 4, the affinity for the contaminant is lowered by the methylene group included in Formulae (3-2) and (3-4), and the fluorine-containing ether compound having high chemical substance resistance is obtained.

In Formula (1), it is still more preferable that R² represents a linking group represented by Formula (3-1) or (3-2) and R⁵ represents a linking group represented by Formula (3-3) or (3-4).

In the fluorine-containing ether compound represented by Formula (1), R² and R⁵ may be the same as or different from each other. Further, the expression “R² and R⁵ may be the same as each other” denotes that the atom included in R² and the atom included in R⁵ are symmetrically disposed with respect to —CH₂—R³[—CH₂—R⁴—CH₂—R³]ₙ—CH₂—. That is, it is preferable that “R² is represented by Formula (3-1) and R⁵ is represented by Formula (3-3), and p1 in Formula (3-1) and p2 in Formula (3-3) are the same as each other” or “R² is represented by Formula (3-2) and R⁵ is represented by Formula (3-4), and q1 in Formula (3-2) and r2 in Formula (3-4) are the same as each other”.

### (Divalent Linking Group Represented by R⁴)

In the fluorine-containing ether compound represented by Formula (1), R⁴ represents a divalent linking group having one or more polar groups, which is disposed between the PFPE chains represented by R³ in a case where n represents 1 or 2. The polar group included in R⁴ causes the fluorine-containing ether compound and the protective layer to adhere to each other. Therefore, a lubricating layer with a small thickness can be formed at a sufficient coating rate by using a lubricant containing the fluorine-containing ether compound represented by Formula (1).

Examples of the polar group included in R⁴ include a hydroxy group (—OH), an amino group (—NH₂), a carboxy group (—COOH), a formyl group (—((C═O)H)), a carbonyl group (—CO—), and a sulfo group (—SO₃H). The number of polar groups included in R⁴ is preferably 1 to 3 and more preferably 1 or 2. In a case where R⁴ has two or more polar groups, the kinds of the polar groups may be partially or entirely the same as or different from each other.

It is preferable that R⁴ includes a hydroxy group as a polar group and that all the polar groups included in R⁴ are hydroxy groups. The hydroxy group has a large interaction with a protective layer, particularly a protective layer formed of a carbon-based material. Therefore, in a case where R⁴ includes a hydroxy group as a polar group, the lubricating layer containing the fluorine-containing ether compound has high adhesion to the protective layer.

It is preferable that oxygen atoms are disposed at both ends of R⁴. The oxygen atoms disposed at both ends of the linking group form a methylene group (—CH₂—) and an ether bond (—O—) disposed on both sides thereof. These two ether bonds impart moderate flexibility to the fluorine-containing ether compound represented by Formula (1), and increase the affinity between the polar group included in the linking group and the protective layer.

R⁴ represents preferably an alkylene group having oxygen atoms at both ends and 3 to 6 carbon atoms, in which one or two hydrogen atoms are substituted with a hydroxy group and more preferably an alkylene group having 3 or 4 carbon atoms. The alkylene group may have an ether bond between carbon atoms.

Specifically, it is preferable that n pieces of R⁴'s each independently represent a linking group represented by Formula (4). The reason for this is that in a case where R⁴ represents a linking group represented by Formula (4), the structure between the PFPE chains is flexible, and the interaction with the protective layer is further enhanced.

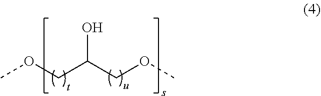

(In Formula (4) s represents an integer of 1 to 3. t represents an integer of 1 to 5.) u represents an integer of 1 to 5. In a case where s represents 2 or greater, combinations of t and u in each structural unit may be the same as or different from each other. In Formula (4), the oxygen atom at the end on the left side is bonded to a group disposed on the R¹ side of R⁴ in Formula (1). In Formula (4), the oxygen atom at the end on the right side is bonded to a group disposed on the R⁶ side of R⁴ in Formula (1).)

In the linking group represented by Formula (4), s represents an integer of 1 to 3. Since s represents 1 or greater, the linking group represented by R⁴ has 1 or more hydroxy groups, and thus a fluorine-containing ether compound capable of obtaining a lubricating layer with satisfactory adhesion to the protective layer is obtained. In addition, since s represents 3 or less, the number of hydroxy groups in the linking group represented by R⁴ is large, and thus the polarity of the fluorine-containing ether compound is not extremely high so that occurrence of contamination due to the chemical substance can be prevented.

In the linking group represented by Formula (4), it is preferable that s represents 1 or 2. In a case where s represents 1 or 2, the number of hydroxy groups in the linking group represented by R⁴ is appropriate, and hydroxy groups that are not involved in bonding with the active points on the protective layer are less likely to take in contaminants so that a lubricating layer having satisfactory chemical substance resistance can be formed.

In the linking group represented by Formula (4), t and u each represent an integer of 1 to 5. Since t and u represent an integer of 1 to 5, the number of carbon atoms in the molecule is not extremely increased by the methylene group included in Formula (4). Therefore, the fluorine-containing ether compound represented by Formula (1) is likely to have an appropriate proportion of fluorine atoms in the entire molecule, and thus a lubricating layer having excellent lubricity can be formed. It is preferable that t and u each represent 1 to 4.

In a case where s in Formula (4) represents 2 or greater, combinations of t and u in each structural unit (—(CH₂)ₜ—CH(OH)—(CH₂)ᵤ—O—) may be the same as or different from each other. In each structural unit, it is preferable that at least one of t or u represents 1.

It is more preferable that n pieces of R⁴'s are each independently represented by any of Formulae (4-1) to (4-3). It is preferable that R⁴ is represented by any of Formulae (4-1) to (4-3) from the viewpoint that the fluorine-containing ether compound containing this linking group is easily synthesized.

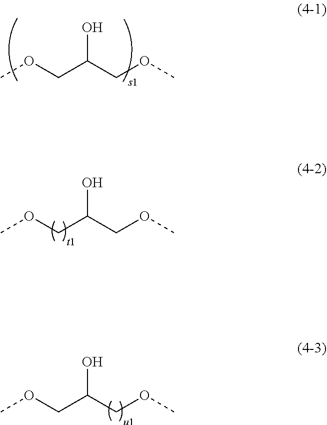

(s1 in Formula (4-1) represents an integer of 1 to 3.)

(t1 in Formula (4-2) represents an integer of 2 to 4. In Formula (4-2), an oxygen atom at an end on a left side is bonded to a methylene group adjacent to an R¹ side of R⁴ in Formula (1).)

(u1 in Formula (4-3) represents an integer of 2 to 4. In Formula (4-3), an oxygen atom at an end on a right side is bonded to a methylene group adjacent to an R⁶ side of R⁴.)

In the linking group represented by Formula (4-1), s1 represents an integer of 1 to 3. Since s1 represents 1 or greater, the fluorine-containing ether compound is capable of obtaining a lubricating layer with satisfactory adhesion to the protective layer. In addition, since s1 represents 3 or less, the number of hydroxy groups in the linking group represented by R⁴ is large, and thus the polarity of the fluorine-containing ether compound is not extremely high so that occurrence of contamination due to the chemical substance can be prevented.

In the linking group represented by Formula (4-1), in a case where s1 in Formula (4-1) represents 2 or 3, the distance between the hydroxy groups included in the linking group represented by Formula (4-1) is appropriate. As a result, even in a case where a plurality of hydroxy groups included in R⁴ are present, the hydroxy groups included in R⁴ are each likely to be involved in the bonding to the active points on the protective layer. It is preferable that s1 represents 1 or 2.

In the linking group represented by Formulae (4-2) and (4-3), t1 and u1 each represent an integer of 2 to 4. Since t1 and u1 represent an integer of 2 to 4, the affinity for the contaminant is lowered by the methylene group included in Formulae (4-2) and (4-3), and the fluorine-containing ether compound having high chemical substance resistance is obtained.

In a case where n represents 2, two R⁴'s in Formula (1) may be the same as or different from each other. Further, the expression “two R⁴'s are the same as each other” denotes that the atom included in R⁴ on the R¹ side and the atom included in R⁴ on the R⁶ side are symmetrically disposed with respect to R³ at the center of the molecule. That is, it is preferable that “two R⁴'s are represented by Formula (4-1) and s1's in Formula (4-1) are the same as each other” or “one of two R²'s is represented by Formula (4-2) and the other is represented by Formula (4-3), and t1's in Formula (4-2) and u1's in Formula (4-3) are the same as each other”.

It is preferable that the polar groups in R², R⁴, and R⁵ in Formula (1) are all hydroxy groups. In a case where n represents 0, the above-described polar group denotes a polar group in R² and R⁵. This is because, in a case where all the polar groups in R², R⁴, and R⁵ are hydroxy groups, the state of coating the protective layer is uniform.

In a case where some or all of the polar groups in R², R⁴, and R⁵ in Formula (1) are hydroxy groups, the total number of the hydroxy groups included in R², the hydroxy groups included in R⁴, and the hydroxy groups included in R⁵ is preferably 2 to 8 and more preferably 2 to 6. The total number described above denotes the total number of the hydroxy group included in R² and the hydroxy group included in R⁵ in a case where n represents 0. In a case where the total number of the above-described hydroxy groups is 2 or more, the interaction between the hydroxy groups contained in R² and R⁵ and the protective layer is more effectively obtained. As a result, the fluorine-containing ether compound can form a lubricating layer having higher adhesion to the protective layer. In addition, in a case where the total number of hydroxy groups is 8 or less, since the hydroxy group which is not involved in the bonding between the lubricating layer and the active point on the protective layer can prevent the environmental substance which generates the contaminant from being attracted to the lubricating layer, the contamination due to the chemical substances is suppressed.

It is preferable that at least one of R², R⁴, or R⁵ has a glycerin structure (—OCH₂CH(OH)CH₂O—). Since the glycerin structure is flexible, the hydroxy groups can each independently move, and the interaction between the hydroxy groups and the protective layer is more strengthened. In this manner, a lubricating layer with more suppressed pickup can be formed.

### (PFPE Chain Represented by R³)

In the fluorine-containing ether compound represented by General Formula (1), R³ represents a perfluoropolyether chain. In a case where the lubricant containing the fluorine-containing ether compound according to the present embodiment is applied onto the protective layer to form a lubricating layer, the surface of the protective layer is coated with the PFPE chain represented by R³ and the lubricity is imparted to the lubricating layer, and thus the frictional force between the magnetic head and the protective layer is reduced. The PFPE chain represented by R³ is appropriately selected according to the performance required for the lubricant containing the fluorine-containing ether compound.

(n+1) pieces of R³'s may be partially or entirely the same as or different from each other. It is preferable that (n+1) pieces of R's are entirely the same as each other. This is because the state of coating the protective layer with the fluorine-containing ether compound is more uniform and thus a lubricating layer having more satisfactory adhesion is formed. The case where two or more R³'s among (n+1) pieces of R³'s are the same as each other denotes that (n+1) pieces of R³'s include two or more R³'s having the same structure of the repeating unit of the PFPE chain. R³'s that are the same as each other also include R³'s having the same repeating unit but having different average degree of polymerizations.

Examples of the PFPE chain represented by R³ include a polymer or a copolymer of perfluoroalkylene oxide. Examples of the perfluoroalkylene oxide include perfluoromethylene oxide, perfluoroethylene oxide, perfluoro-n-propylene oxide, perfluoroisopropylene oxide, and perfluorobutylene oxide.

It is preferable that (n+1) pieces of R³'s in Formula (1) each independently represent a PFPE chain represented by Formula (5) derived from, for example, a polymer or a copolymer of perfluoroalkylene oxide.

—(CF₂)_{w1}—O—(CF₂O)_{w2}—(CF₂CF₂O)_{w3}—(CF₂CF₂CF₂O)_{w4}—(CF₂CF₂CF₂CF₂O)_{w5}—(CF₂)_{w6}— (5)

(In Formula (5), w2, w3, w4, and w5 represent an average degree of polymerization, and each independently represent 0 to 20. Here, all of w2, w3, w4, and w5 do not represent 0 at the same time. w1 and w6 represent an average value representing the number of CF₂'s, and each independently represent 1 to 3. The arrangement order of (CF₂O), (CF₂CF₂O), (CF₂CF₂CF₂O), and (CF₂CF₂CF₂CF₂O), which are the repeating units in Formula (5), is not particularly limited.)

In Formula (5), w2, w3, w4, and w5 represent an average degree of polymerization, and each independently represent 0 to 20, preferably 0 to 15, and more preferably 0 to 10.

In Formula (5), w1 and w6 represent the average value indicating the number of CF₂'s, and each independently represent 1 to 3. w1 and w6 are determined depending on the structure of the repeating unit disposed at the end of the chain structure in the PFPE chain represented by Formula (5).

(CF₂O), (CF₂CF₂O), (CF₂CF₂CF₂O), and (CF₂CF₂CF₂CF₂O) in Formula (5) are repeating units. The arrangement order of the repeating units in Formula (5) is not particularly limited. In addition, the number of kinds of repeating units in Formula (5) is also not particularly limited.

It is preferable that (n+1) pieces of R³'s in Formula (1) each independently represent any one selected from PFPE chains represented by Formulae (5-1) to (5-4).

In a case where (n+1) pieces of R³'s are any one selected from the PFPE chains represented by Formulae (5-1) to (5-4), the fluorine-containing ether compound capable of obtaining a lubricating layer having satisfactory lubricity is obtained. In addition, in a case where (n+1) pieces of R³'s are any one selected from the PFPE chains represented by Formulae (5-1) to (5-4), the ratio of the number of oxygen atoms (number of ether bonds (—O—)) to the number of carbon atoms in the PFPE chain is appropriate. Therefore, the fluorine-containing ether compound has moderate hardness. Therefore, the fluorine-containing ether compound applied onto the protective layer is unlikely to be aggregated on the protective layer, and a lubricating layer having a thinner thickness can be formed with a sufficient coating rate. In addition, in a case where (n+1) pieces of R³'s are any one selected from the PFPE chains represented by Formulae (5-1) to (5-4), the fluorine-containing ether compound has moderate flexibility, and thus is capable of forming a lubricating layer with more satisfactory chemical substance resistance is obtained.

—CF₂—(OCF₂CF₂)ₕ—(OCF₂)ᵢ—OCF₂— (5-1)

(In Formula (5-1), h and i represent an average degree of polymerization, where h represents 1 to 20 and i represents 0 to 20.)

—CF₂CF₂—(OCF₂CF₂CF₂)ⱼ—OCF₂CF₂— (5-2)

(In Formula (5-2), j represents an average degree of polymerization, and represents 1 to 15.)

—CF₂CF₂CF₂—(OCF₂CF₂CF₂CF₂)ₖ—OCF₂CF₂CF₂— (5-3)

(In Formula (5-3), k represents an average degree of polymerization, and represents 1 to 10.)

—(CF₂)_{w7}—O—(CF₂CF₂CF₂O)_{w8}—(CF₂CF₂O)_{w9}—(CF₂)_{w10}— (5-4)

(In Formula (5-4), w8 and w9 represent an average degree of polymerization, and each independently represents 1 to 20.) w7 and w10 represent an average value representing the number of CF₂'s, and each independently represent 1 to 2.)

In Formula (5-1), the arrangement order of (OCF₂CF₂) and (OCF₂) which are the repeating units is not particularly limited. In Formula (5-1), the number h of (OCF₂CF₂)'s and the number i of (OCF₂)'s may be the same as or different from each other. The PFPE chain represented by Formula (5-1) may be a polymer of (OCF₂CF₂). In addition, the PFPE chain represented by Formula (5-1) may be any of a random copolymer, a block copolymer, or an alternating copolymer consisting of (OCF₂CF₂) and (OCF₂).

In Formulae (5-1) to (5-3), since h indicating the average degree of polymerization represents 1 to 20, i represents 0 to 20, j represents 1 to 15, and k represents 1 to 10, a fluorine-containing ether compound capable of obtaining a lubricating layer having satisfactory lubricity is obtained. In addition, in Formulae (5-1) to (5-3), since h and i indicating the average degree of polymerization represent 20 or less, j represents 15 or less, and k represents 10 or less, the viscosity of the fluorine-containing ether compound is not extremely high, and a lubricant containing the fluorine-containing ether compound is easy to apply, which is preferable. h, i, j, and k indicating the average degree of polymerization represent preferably 1 to 10, more preferably 1.5 to 8, and still more preferably 2 to 7 from the viewpoint that the fluorine-containing ether compound that is easily wet and spreads on the protective layer and easily obtains a lubricating layer having a uniform film thickness is obtained.

In Formula (5-4), the arrangement order of (CF₂CF₂CF₂O) and (CF₂CF₂O), which are the repeating units, is not particularly limited. In Formula (5-4), the number w8 of (CF₂CF₂CF₂O)'s and the number w9 of (CF₂CF₂O)'s may be the same as or different from each other. Formula (5-4) may represent any of a random copolymer, a block copolymer, or an alternating copolymer, formed of monomer units (CF₂CF₂CF₂O) and (CF₂CF₂O).

In Formula (5-4), w8 and w9 representing the average degree of polymerization each independently represent 1 to 20, preferably 1 to 15, and more preferably 1 to 10.

w7 and w10 in Formula (5-4) are average values indicating the number of CF₂'s, and each independently represent 1 to 2. w7 and w10 are determined according to the structure of the repeating unit disposed at the end of the chain structure in the PFPE chain represented by Formula (5-4).

(End group represented by R¹ and R⁶)

In the fluorine-containing ether compound represented by Formula (1), Each R¹ and R⁶ represents an organic group having 1 to 50 carbon atoms, which is bonded to the oxygen atom of R² or R⁵, or a hydrogen atom. At least one of the end groups represented by R¹ and R⁶ is the structure represented by Formula (2) (hereinafter, also referred to as “condensed aromatic group”).

In the fluorine-containing ether compound represented by Formula (1), the condensed aromatic group represented by Formula (2) exhibits a moderate interaction with the protective layer. Therefore, the condensed aromatic group represented by Formula (2) has a function of improving the adhesion between the lubricating layer and the protective layer to form a lubricating layer having satisfactory chemical substance resistance and suppressed pickup.

In Formula (2), Q represents that a carbon atom of a linking group indicated by a dashed arc forms a 5- to 8-membered heterocyclic ring structure together with X and Y The number of ring members in the heterocyclic ring structure is preferably 6.

In Formula (2), one of X and Y represents a carbonyl group (C═O) and the other represents —NH—, —NR⁷— (R⁷ represents a hydrocarbon group having 1 to 8 carbon atoms), or —O—. Therefore, —X—Y— is an amide or an ester. In a case where one of X and Y represents —NR⁷—, examples of R⁷ include an alkyl group, an alkenyl group, an alkynyl group, an aryl group, and an alkyl group substituted with an aryl group, each having 1 to 8 carbon atoms. Specifically, it is preferable that R⁷ represents a methyl group, an ethyl group, a propyl group, a butyl group, an allyl group, a butenyl group, a propargyl group, a phenyl group, or a benzyl group.

Carbon atoms constituting two rings in Formula (2) (here, a carbonyl carbon atom of X or Y is excluded) may have Z which represents a substituent. The number of Z's is 0 to 5, preferably 0 to 3, and more preferably 0 to 2. In a case where Formula (2) has two or more Z's, the kinds of Z's may be partially or entirely the same as or different from each other.

Z's each independently represent an organic group having 1 to 8 carbon atoms. The organic group is preferably selected from the group consisting of an alkyl group, an alkenyl group, an alkynyl group, an aryl group, an alkyl group substituted with an aryl group, and an alkoxy group, each having 1 to 8 carbon atoms. Specifically, any of a phenyl group, a methyl group, a methoxy group, an allyl group, a butenyl group, a propargyl group, or a benzyl group is more preferable. In a case where the organic group is any of these groups, the fluorine-containing ether compound in which the interaction between the end group which is R¹ and/or R⁶ and the polar group included in R² and/or R⁵ which is the adjacent linking group is more effectively suppressed and which has a high affinity for the protective layer is obtained.

In a case where two or more of Z's are present, two Z's may be bonded to each other to form a ring structure. In this case, it is preferable that two Z's are each bonded to two adjacent carbon atoms which are not carbon atoms included in —X—Y— without being bonded to R² or R⁵ among the carbon atoms constituting two rings. The ring structure is preferably a 5-membered ring or a 6-membered ring and more preferably a 6-membered ring. The ring structure may be formed of only carbon atoms or may include a heteroatom (for example, —O— or —NH—). In a case where two Z's are bonded to each other to form a ring structure, the ring structure is preferably a benzene ring.

Any of carbon atoms constituting two rings in Formula (2) (here, the carbonyl carbon atom of X or Y is excluded) is bonded to the oxygen atom at the end of R² or R⁵. That is, between two rings, a carbon atom constituting the benzene ring may be bonded to R² or R⁵, and a carbon atom constituting the heterocyclic ring structure represented by Q may be bonded to R² or R⁵. The carbon atom included in Z is not included in the carbon atoms constituting the two rings.

The condensed aromatic group represented by Formula (2) is preferably a structure represented by Formula (2-1).

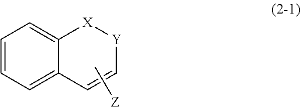

(In Formula (2-1), one of X and Y represents a carbonyl group (C═O), and the other represents —NH—, —NR⁷— (R⁷ represents a hydrocarbon group having 1 to 8 carbon atoms), or —O—. Z represents 0 to 5 substituents. Z's each independently represent an organic group having 1 to 8 carbon atoms. Z may be bonded to any of carbon atoms constituting two rings in Formula (2-1) (where a carbonyl carbon atom of X or Y is excluded). In a case where two or more Z's are present, two Z's may be bonded to each other to form a ring structure. Any of the carbon atoms constituting the two rings in Formula (2-1) (where a carbonyl carbon atom of X or Y is excluded) is bonded to the oxygen atom at the end of R² or R⁵.)

Formula (2-1) represents a structure in which Q in Formula (2) represents a 6-membered heterocyclic ring and the position of X is a position adjacent to a carbon atom shared by the heterocyclic ring and the benzene ring. It is preferable that at least one of R¹ or R⁶ is represented by Formula (2-1) from the viewpoint of facilitating the production of the fluorine-containing ether compound.

Specific examples and preferred examples of each structure of X, Y, and Z, the number of Z's, and the bonding form of Z in Formula (2-1) are the same as described in the section of Formula (2).

Examples of the condensed aromatic group represented by Formula (2-1) include any of the following organic groups represented by Formulae (6-1) to (6-11), but the condensed aromatic group is not limited to the organic groups described below. The dotted line in Formulae (6-1) to (6-11) is a bonding site bonded to R² or R⁵ in Formula (1).

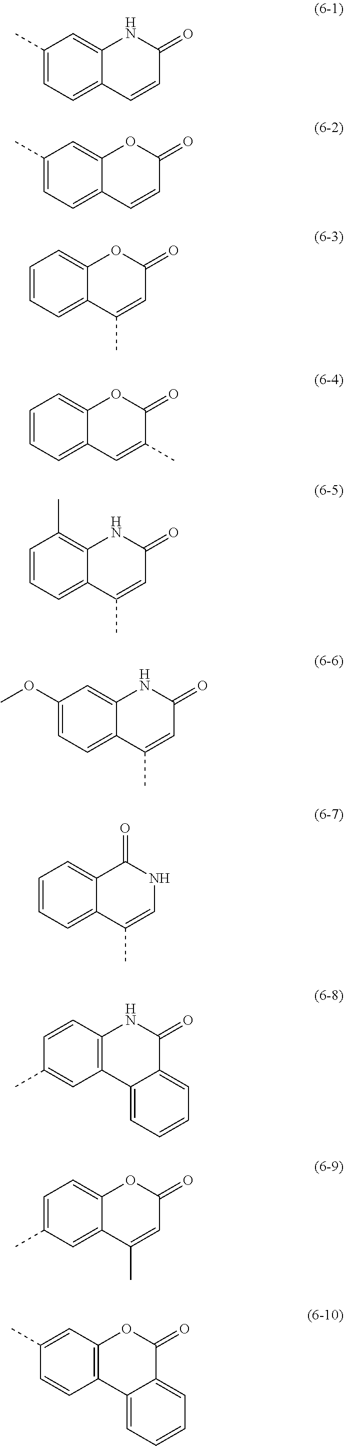

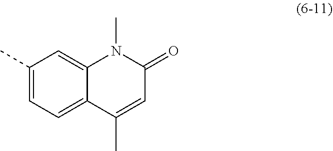

Among Formulae (6-1) to (6-11), it is preferable to use Formulae (6-1) to (6-3) from the viewpoint that a fluorine-containing ether compound is relatively easily produce.

In the fluorine-containing ether compound represented by Formula (1), in a case where both end groups represented by R¹ and R⁶ are the condensed aromatic groups represented by Formula (2), R¹ and R⁶ may be the same as or different from each other. It is preferable that R¹ and R⁶ are the same as each other from the viewpoint that the fluorine-containing ether compound is easy to produce.

In a case where, among the end groups represented by R¹ and R⁶, only one end group (for example, R¹) has the structure represented by Formula (2), the other end group (for example, R⁶) may be an organic group having 1 to 50 carbon atoms or a hydrogen atom. In a case where the other end group is the organic group, the number of carbon atoms included in the organic group is preferably 2 to 20 and more preferably 2 to 10. It is preferable that the other end group is an organic group having at least one double bond or triple bond, which is not a structure represented by Formula (2), an alkyl group having 1 to 8 carbon atoms, which may have a substituent, or a hydrogen atom.

In a case where the other end group is an organic group having at least one double bond or triple bond, which is not a structure represented by Formula (2), examples thereof include a group having an aromatic ring, a group having an unsaturated heterocyclic ring, a group including an alkenyl group, and a group including an alkynyl group. The double bond and the triple bond may be a carbon-carbon bond or a bond between a carbon atom and a heteroatom.

In a case where the other end group is an alkyl group having 1 to 8 carbon atoms, which may have a substituent, examples of the substituent include an alkoxy group, a hydroxy group, a mercapto group, a carboxy group, a carbonyl group, an amino group, and a fluoro group. The alkyl group has more preferably 1 to 6 carbon atoms and still more preferably 1 to 4 carbon atoms.

The end group is preferably an alkyl group having 1 to 8 carbon atoms and a hydroxy group, and the hydroxy group is more preferably a primary hydroxy group.

In a case where the other end group is a hydrogen atom, the hydrogen atom as R⁶ and the oxygen atom (or the hydrogen atom as R¹ and the oxygen atom disposed at the end of R²) disposed at the end of R⁵ form a hydroxy group.

In a case where the other end group is an organic group having at least one double bond or triple bond, which is not the structure represented by Formula (2), specific examples thereof include a phenyl group, a methoxyphenyl group, a fluorinated phenyl group, a naphthyl group, a phenethyl group, a methoxyphenethyl group, a fluorinated phenethyl group, a benzyl group, a methoxybenzyl group, a naphthylmethyl group, a methoxynaphthyl group, a pyrrolyl group, a pyrazolyl group, a methylpyrazolylmethyl group, an imidazolyl group, a furyl group, a furfuryl group, an oxazolyl group, an isoxazolyl group, a thienyl group, a thienylethyl group, a thiazolyl group, a methylthiazolylethyl group, an isothiazolyl group, a pyridyl group, a pyrimidinyl group, a pyridazinyl group, a pyrazinyl group, an indolinyl group, a benzofuranyl group, a benzothienyl group, a benzimidazolyl group, a benzoxazolyl group, a benzothiazolyl group, a benzopyrazolyl group, a benzoisoxazolyl group, a benzoisothiazolyl group, a quinolyl group, an isoquinolyl group, a quinazolinyl group, a quinoxalinyl group, a phthalazinyl group, a cinnolinyl group, a vinyl group, an allyl group, a butenyl group, a propynyl group, a propargyl group, a butynyl group, a methylbutynyl group, a pentynyl group, a methylpentynyl group, a hexynyl group, and a cyanoethyl group.

In a case where the other end group is an alkyl group having 1 to 8 carbon atoms, which may have a substituent, specific examples thereof include a methyl group, an ethyl group, a propyl group, a butyl group, a pentyl group, a hexyl group, an octyl group, a trifluoromethyl group, a perfluoroethyl group, a perfluoropropyl group, a perfluorobutyl group, a perfluoropentyl group, a perfluorohexyl group, an octafluoropentyl group, a tridecafluorooctyl group, a hydroxymethyl group (—CH₂OH), a hydroxyethyl group (—CH₂CH₂OH), a hydroxypropyl group (—CH₂CH₂CH₂OH), and a hydroxybutyl group (—CH₂CH₂CH₂CH₂OH).

Among the examples described above, the other end group is preferably any of a phenyl group, a methoxyphenyl group, a naphthyl group, a phenethyl group, a methoxyphenethyl group, a fluorinated phenethyl group, a thienylethyl group, an allyl group, a butenyl group, a propargyl group, or a hydroxyethyl group and particularly preferably any of a phenyl group, a naphthyl group, a thienylethyl group, an allyl group, a butenyl group, or a hydroxyethyl group. In this case, the fluorine-containing ether compound capable of forming a lubricating layer having more excellent wear resistance is obtained.

In a case where both R¹ and R⁶ represent an end group represented by Formula (2), the numbers of polar groups in R² and R⁵ are each preferably 1. In this case, since the polar groups included in R² and R⁵ can be involved in the bonding of the lubricating layer to the active point on the protective layer, more satisfactory chemical substance resistance can be obtained.

It is preferable that (n+1) pieces of PFPE chains represented by R³ are the same as each other, and R¹—R²— and R⁶—R⁵— are the same as each other from the viewpoint that the fluorine-containing ether compound represented by Formula (1) can be easily and effectively produced. In addition, the expression “(n+1) pieces of PFPE chains represented by R³ are the same as each other, and R¹—R²— and R⁶—R⁵— are the same as each other” denotes that R¹—R²— and R⁶—R⁵— are the same as each other in a case where n represents 0, and all R³'s in the molecule are the same as each other and R¹—R²— and R⁶—R⁵— are the same as each other in a case where n represents 1 or 2.

It is more preferable that n represents 1 or 2 and R⁴ represents a linking group represented by Formula (4-1) from the viewpoint that the fluorine-containing ether compound can be easily and effectively produced. In addition, since such a fluorine-containing ether compound has a symmetrical structure with R⁴ as the center in a case where n represents 1 and has a symmetrical structure with R³ as the center in the molecule in a case where n represents 2, a lubricating layer that is easy to uniformly wet on the protective layer and has a uniform film thickness is easily obtained, which is preferable.

The fluorine-containing ether compound represented by Formula (1) is specifically preferably any of compounds represented by Formulae (A) to (Z), (AA), and (AB). In a case where the compound represented by Formula (1) is any of compounds represented by Formulae (A) to (Z), (AA), or (AB), a raw material is easily available, and a lubricating layer having excellent adhesion even in a case where the thickness thereof is small and capable of obtaining more excellent chemical substance resistance and an excellent pickup suppression effect can be formed, which is preferable.

In all of the compounds represented by Formulae (A) to (Z), (AA), and (AB), (n+1) pieces of PFPE chains represented by R³ in Formula (1) are all the same as each other. In the compounds represented by Formulae (A) to (Z), (AA), and (AB), Rf₁, Rf₂, and Rf₃ representing a PFPE chain each have the following structure. Further, since h, i, j, and k in Formulae (A) to (Z), (AA), and (AB) represent a value indicating the average degree of polymerization, these values are not necessarily integers.

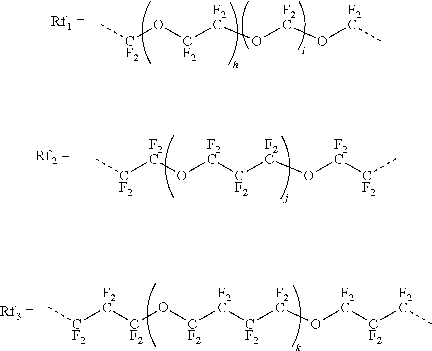

In the compounds represented by Formulae (A) to (F), n represents 0, R⁶ represents a hydroxyethyl group, R² represents a linking group represented by Formula (3-1), p1 represents 1, R⁵ represents a linking group represented by Formula (3-3), p2 represents 1, and R³ is represented by Formula (5-1).

In the compound represented by Formula (A), R¹ represents an organic group represented by Formula (6-1).

In the compound represented by Formula (B), R¹ represents an organic group represented by Formula (6-2).

In the compound represented by Formula (C), R¹ represents an organic group represented by Formula (6-3).

In the compound represented by Formula (D), R¹ represents an organic group represented by Formula (6-6).

In the compound represented by Formula (E), R¹ represents an organic group represented by Formula (6-7).

In the compound represented by Formula (F), R¹ represents an organic group represented by Formula (6-10).

In the compound represented by Formula (G) or (H), n represents 0, R² represents a linking group represented by Formula (3-1), p1 represents 1, R⁵ represents a linking group represented by Formula (3-3), p2 represents 1, and R³ is represented by Formula (5-1).

In the compound represented by Formula (G), R¹ and R⁶ represent an organic group represented by Formula (6-1).

In the compound represented by Formula (H), R¹ and R⁶ represent an organic group represented by Formula (6-2).

In the compound represented by Formulae (I) and (J), n represents 0, R² represents a linking group represented by Formula (3-1), p1 represents 1, and R³ is represented by Formula (5-1).

In the compound represented by Formula (I), R¹ represents an organic group represented by Formula (6-1), R⁶ represents an allyl group, R⁵ represents a linking group represented by Formula (3-3), and p2 represents 2.

In the compound represented by Formula (J), R¹ represents an organic group represented by Formula (6-2), R⁶ represents a phenyl group, R⁵ represents a linking group represented by Formula (3-3), and p2 represents 1.

In the compound represented by Formulae (K) and (L), n represents 0, R⁶ represents a hydroxyethyl group, R⁵ represents a linking group represented by Formula (3-3), p2 represents 1, and R³ is represented by Formula (5-1).

In the compound represented by Formula (K), R¹ represents an organic group represented by Formula (6-1), R² represents a linking group represented by Formula (3-1), and p1 represents 2.

In the compound represented by Formula (L), R¹ represents an organic group represented by Formula (6-2), R² represents a linking group represented by Formula (3-2), and q1 represents 2.

In the compounds represented by Formulae (M) and (N), n represents 0, R⁶ represents a hydroxyethyl group, R² represents a linking group represented by Formula (3-1), p1 represents 1, R⁵ represents a linking group represented by Formula (3-3), and p2 represents 1.

In the compound represented by Formula (M), R¹ represents an organic group represented by Formula (6-1), and R³ is represented by Formula (5-2).

In the compound represented by Formula (N), R¹ represents an organic group represented by Formula (6-2), and R³ is represented by Formula (5-3).

In the compound represented by Formulae (O) and (P), n represents 1, R² represents a linking group represented by Formula (3-1), p1 represents 1, R⁵ represents a linking group represented by Formula (3-3), p2 represents 1, R⁴ represents a linking group represented by Formula (4-1), s1 represents 1, and R³ is represented by Formula (5-1).

In the compound represented by Formula (O), R¹ and R⁶ represent an organic group represented by Formula (6-1).

In the compound represented by Formula (P), R¹ and R⁶ represent an organic group represented by Formula (6-2).

In the compounds represented by Formulae (Q) and (R), n represents 1, R² represents a linking group represented by Formula (3-1), p1 represents 1, R⁴ represents a linking group represented by Formula (4-1), s1 represents 1, and R³ is represented by Formula (5-1).

In the compound represented by Formula (Q), R represents an organic group represented by Formula (6-1), R⁶ represents a hydroxyethyl group, R⁵ represents a linking group represented by Formula (3-3), and p2 represents 1.

In the compound represented by Formula (R), R¹ represents an organic group represented by Formula (6-2), R⁶ represents an allyl group, R⁵ represents a linking group represented by Formula (3-3), and p2 represents 2.

In the compound represented by Formulae (S) and (T), n represents 1, s1 represents 1, R⁴ represents a linking group represented by Formula (4-1), and R³ is represented by Formula (5-1).

In the compound represented by Formula (S), R¹ and R⁶ represents an organic group represented by Formula (6-1), R² represents the linking group represented by Formula (3-1), p1 represents 2, R⁵ represents a linking group represented by Formula (3-3), and p2 represents 2.

In the compound represented by Formula (T), R¹ and R⁶ represents an organic group represented by Formula (6-2), R² represents a linking group represented by Formula (3-2), q1 represents 2, and R⁵ represents a linking group represented by Formula (3-4), and r2 represents 2.

In the compounds represented by Formulae (U) and (V), n represents 1, R¹ and R⁶ represents an organic group represented by Formula (6-1), R² represents a linking group represented by Formula (3-1), p1 represents 1, R⁵ represents a linking group represented by Formula (3-3), and p2 represents 1.

In the compound represented by Formula (U), R⁴ represents a linking group represented by Formula (4-3), u1 represents 2, and R³ is represented by Formula (5-1).

In the compound represented by Formula (V), R⁴ represents a linking group represented by Formula (4-1), s1 represents 1, and R³ is represented by Formula (5-2).

In the compounds represented by Formulae (W) to (Y), n represents 2, R² represents a linking group represented by Formula (3-1), p1 represents 1, R⁵ represents a linking group represented by Formula (3-3), p2 represents 1, R⁴ represents a linking group represented by Formula (4-1), s1 represents 1, and R³ is represented by Formula (5-1).

In the compound represented by Formula (W), R¹ and R⁶ represent an organic group represented by Formula (6-1).

In the compound represented by Formula (X), R¹ and R⁶ represent an organic group represented by Formula (6-2).

In the compound represented by Formula (Y), R¹ represents an organic group represented by Formula (6-1), and R⁶ represents a hydroxyethyl group.

In the compound represented by Formulae (Z), (AA), and (AB), n represents 2, and R¹ and R⁶ represent an organic group represented by Formula (6-1).

In the compound represented by Formula (Z), R² represents a linking group represented by Formula (3-1), p1 represents 2, R⁵ represents a linking group represented by Formula (3-3), p2 represents 2, R⁴ represents a linking group represented by Formula (4-1), s1 represents 1, and R³ is represented by Formula (5-1).

In the compound represented by Formula (AA), R² represents a linking group represented by Formula (3-1), p1 represents 1, R⁵ represents a linking group represented by Formula (3-3), p2 represents 1, R⁴ on the R¹ side represents a linking group represented by Formula (4-3), u1 represents 2, R⁴ on the R⁶ side represents a linking group represented by Formula (4-2), t1 represents 2, and R³ is represented by Formula (5-1).

In the compound represented by Formula (AB), R² represents a linking group represented by Formula (3-1), p1 represents 1, R⁵ represents a linking group represented by Formula (3-3), p2 represents 1, R⁴ represents a linking group represented by Formula (4-1), s1 represents 1, and R³ is represented by Formula (5-2).

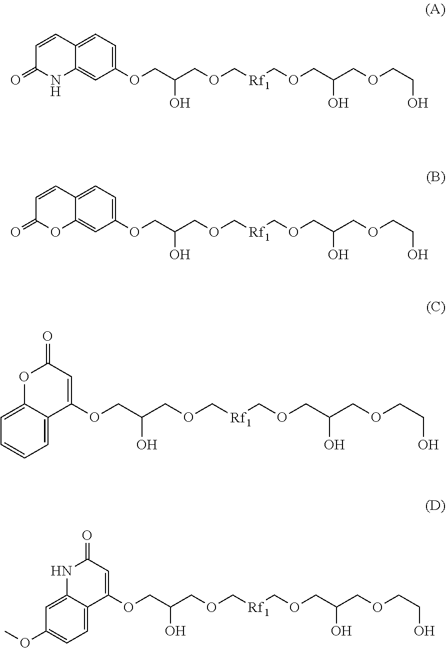

(In Rf₁ in Formula (A), h and i represent an average degree of polymerization, where h represents 1 to 20 and i represents 0 to 20.)

(In Rf₁ in Formula (B), h and i represent an average degree of polymerization, where h represents 1 to 20 and i represents 0 to 20.)

(In Rf₁ in Formula (C), h and i represent an average degree of polymerization, where h represents 1 to 20 and i represents 0 to 20.)

(In Rf₁ in Formula (D), h and i represent an average degree of polymerization, where h represents 1 to 20 and i represents 0 to 20.)

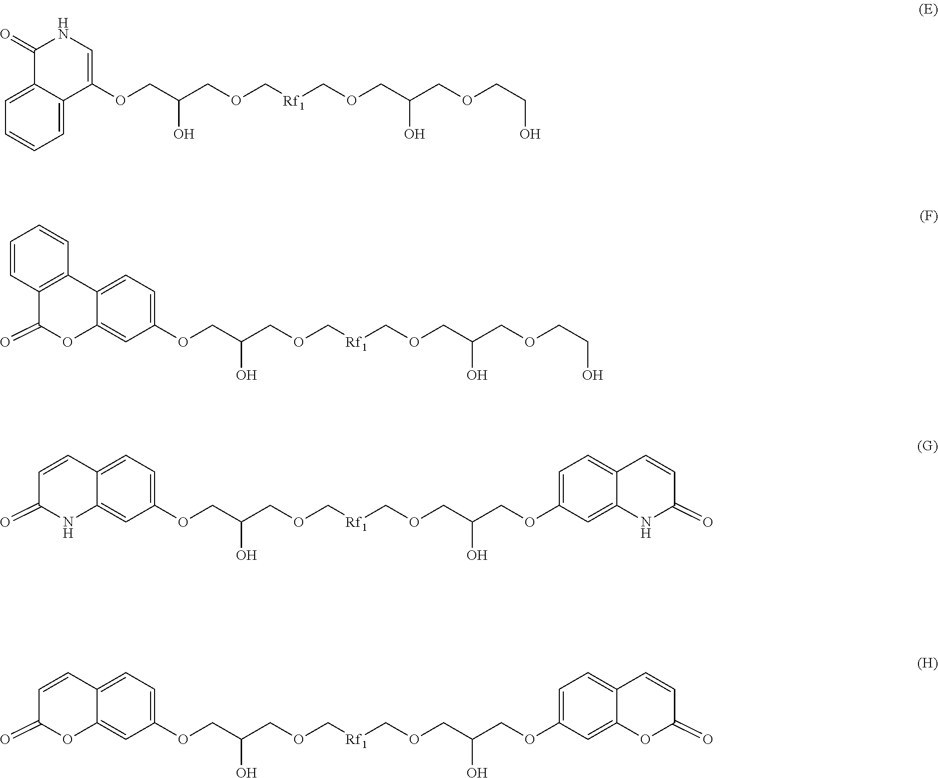

(In Rf₁ in Formula (E), h and i represent an average degree of polymerization, where h represents 1 to 20 and i represents 0 to 20.)

(In Rf₁ in Formula (F), h and i represent an average degree of polymerization, where h represents 1 to 20 and i represents 0 to 20.)

(In Rf₁ in Formula (G), h and i represent an average degree of polymerization, where h represents 1 to 20 and i represents 0 to 20.)

(In Rf₁ in Formula (H), h and i represent an average degree of polymerization, where h represents 1 to 20 and i represents 0 to 20.)

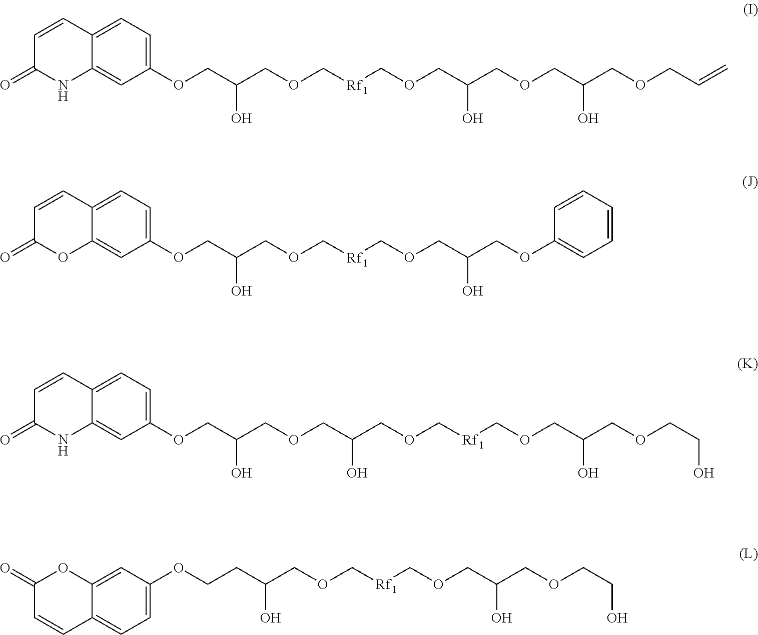

(In Formula (I), Rf₁ represents an average degree of polymerization, where h and i represent an average degree of polymerization, h represents 1 to 20, and i represents 0 to 20.)

(In Rf₁ in Formula (J), h and i represent an average degree of polymerization, where h represents 1 to 20 and i represents 0 to 20.)

(In Rf₁ in Formula (K), h and i represent an average degree of polymerization, where h represents 1 to 20 and i represents 0 to 20.)

(In Rf₁ in Formula (L), h and i represent an average degree of polymerization, where h represents 1 to 20 and i represents 0 to 20.)

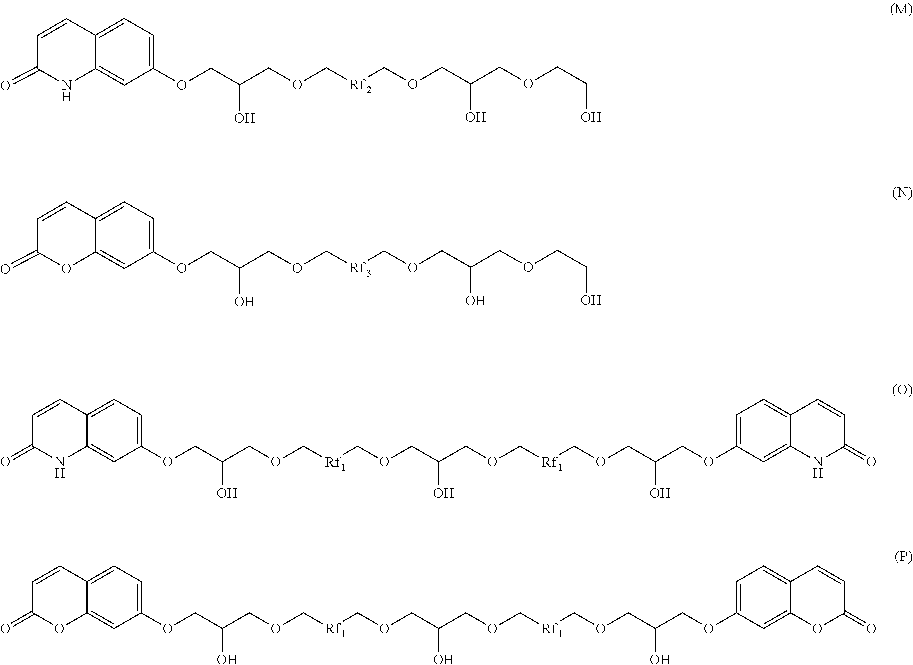

(In Rf₂ in Formula (M), j represents an average degree of polymerization and represents 1 to 15.)

(In Rf₃ in Formula (N), k represents an average degree of polymerization and represents 1 to 10.)

(In two Rf₁'s in Formula (O), h and i represent an average degree of polymerization, where h represents 1 to 20, and i represents 0 to 20. h's and i's in the two Rf₁'s may be the same as or different from each other.)

(In two Rf₁'s in Formula (P), h and i represent an average degree of polymerization, where h represents 1 to 20 and i represents 0 to 20. h's and i's in the two Rf₁'s may be the same as or different from each other.)

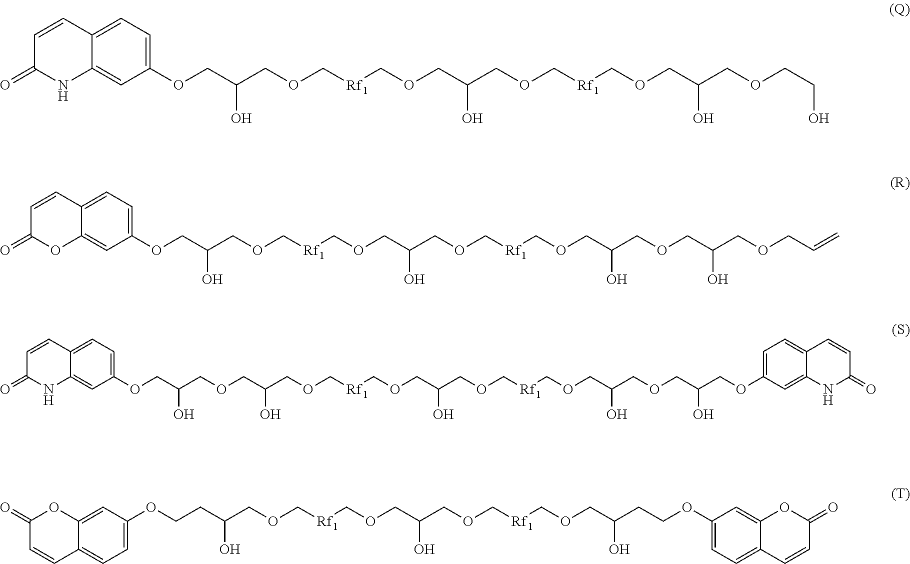

(In two Rf₁'s in Formula (Q), h and i represent an average degree of polymerization, where h represents 1 to 20 and i represents 0 to 20. h's and i's in the two Rf₁'s may be the same as or different from each other.)

(In two Rf₁'s in Formula (R), h and i represent an average degree of polymerization, where h represents 1 to 20 and i represents 0 to 20. h's and i's in the two Rf₁'s may be the same as or different from each other.)

(In two Rf₁'s in Formula (S), h and i represent an average degree of polymerization, where h represents 1 to 20 and i represents 0 to 20. h's and i's in the two Rf₁'s may be the same as or different from each other.)

(In two Rf₁'s in Formula (T), h and i represent an average degree of polymerization, where h represents 1 to 20 and i represents 0 to 20. h's and i's in the two Rf₁'s may be the same as or different from each other.)

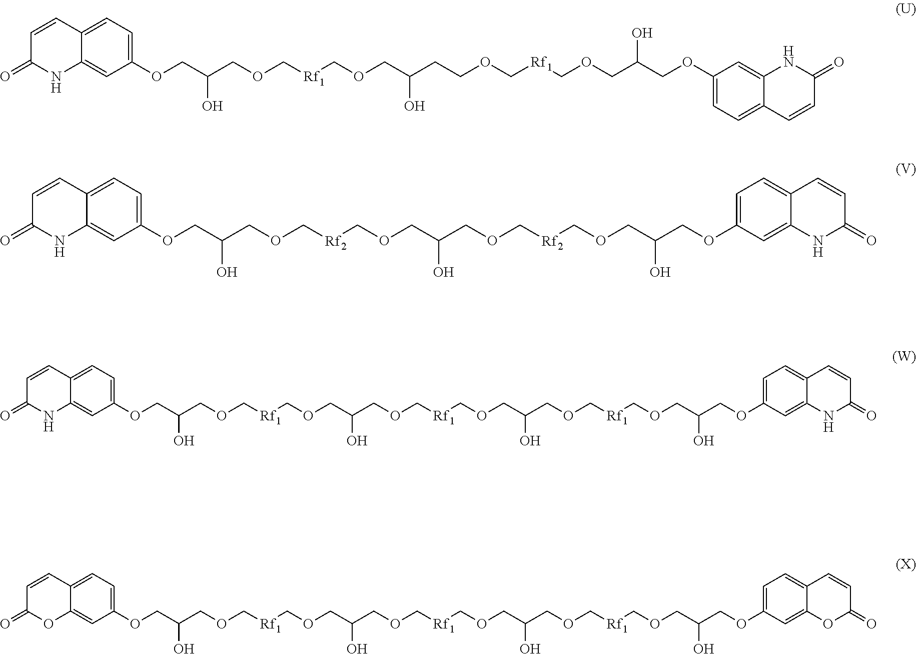

(In two Rf₁'s in Formula (U), h and i represent an average degree of polymerization, where h represents 1 to 20 and i represents 0 to 20. h's and i's in the two Rf₁'s may be the same as or different from each other.)

(In two Rf₂'s in Formula (V), j represents an average degree of polymerization and represents 1 to 15. j's in the two Rf₂'s may be the same as or different from each other.)

(In three Rf₁'s in Formula (W), h and i represent an average degree of polymerization, where h represents 1 to 20 and i represents 0 to 20. h's and i's in the three Rf₁'s may be partially or entirely the same as or different from each other.)

In three Rf₁'s in Formula (X), h and i represent an average degree of polymerization, where h represents 1 to 20 and i represents 0 to 20. h's and i's in the three Rf₁'s may be partially or entirely the same as or different from each other.)

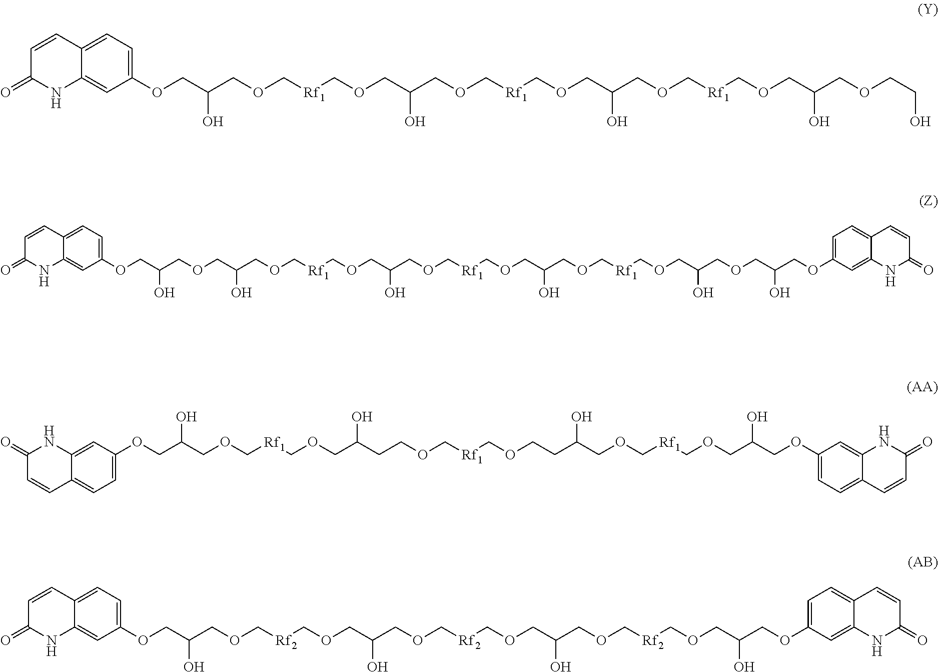

(In three Rf₁'s in Formula (Y), h and i represent an average degree of polymerization, where h represents 1 to 20 and i represents 0 to 20. h's and i's in the three Rf₁'s may be partially or entirely the same as or different from each other.)

(In three Rf₁'s in Formula (Z), h and i represent an average degree of polymerization, where h represents 1 to 20 and i represents 0 to 20. h's and i's in the three Rf₁'s may be partially or entirely the same as or different from each other.)

(In three Rf₁'s in Formula (AA), h and i represent an average degree of polymerization, where h represents 1 to 20 and i represents 0 to 20. h's and i's in the three Rf₁'s may be partially or entirely the same as or different from each other.)

(In three Rf₂'s in Formula (AB), j represents an average degree of polymerization and represents 1 to 15. j's in the three Rf₂'s may be partially or entirely the same as or different from each other.)

The number-average molecular weight (Mn) of the fluorine-containing ether compound of the present embodiment is preferably in a range of 500 to 10,000 and particularly preferably in a range of 1,000 to 5,000. In a case where the number-average molecular weight thereof is 500 or greater, the lubricating layer formed of the lubricant containing the fluorine-containing ether compound according to the present embodiment has excellent heat resistance. The number-average molecular weight of the fluorine-containing ether compound is more preferably 1,000 or greater. In addition, in a case where the number-average molecular weight thereof is 10,000 or less, the viscosity of the fluorine-containing ether compound is appropriate, and a lubricating layer with a small film thickness can be easily formed by applying a lubricant containing the fluorine-containing ether compound. The number-average molecular weight of the fluorine-containing ether compound is preferably 5,000 or less from the viewpoint that the viscosity is easy to handle in a case where the fluorine-containing ether compound is applied to a lubricant.

The number-average molecular weight (Mn) of the fluorine-containing ether compound is a value measured by ¹H-NMR and ¹⁹F-NMR using AVANCE III 400 (manufactured by Bruker BioSpin GmbH). Specifically, the number of repeating units of the PFPE chain is calculated from the integrated value measured by ¹⁹F-NMR, and the number-average molecular weight is determined. In the measurement of nuclear magnetic resonance (NMR), the sample is diluted with a solvent of hexafluorobenzene/d-acetone (4/1 v/v) and measured. The peak of hexafluorobenzene is set to −164.7 ppm as the reference of the ¹⁹F-NMR chemical shift, and the peak of acetone is set to 2.2 ppm as the reference of the ¹H-NMR chemical shift.

In the fluorine-containing ether compound of the present embodiment, it is preferable that the polydispersity (ratio of weight-average molecular weight (Mw) to number-average molecular weight (Mn)) is set to 1.3 or less by fractionating the molecular weight by an appropriate method.

In the present embodiment, a method for fractionating the molecular weight is not particularly limited, and for example, molecular weight fractionation by a silica gel column chromatography method, a gel permeation chromatography (GPC) method, or the like, molecular weight fractionation by a supercritical extraction method, and the like can be used.

### “Production Method”

A method for producing the fluorine-containing ether compound of the present embodiment is not particularly limited, and the fluorine-containing ether compound can be produced by a known production method of the related art. The fluorine-containing ether compound of the present embodiment can be produced, for example, by the production methods [1] to [3] described below.

[1] Production Method in Case of n=0 in Formula (1)
[1-1] in Case where R¹—R²— and R⁶—R⁵— are the Same as Each Other

First, a fluorine-based compound in which a hydroxymethyl group (—CH₂OH) is disposed at both ends of a perfluoropolyether chain corresponding to R³ in Formula (1) is prepared.

Next, the hydroxy group of the hydroxymethyl group disposed at one end of the fluorine-based compound reacts with the epoxy group of the epoxy compound having a group represented by R¹—R²— in Formula (1) (=group as R⁶—R⁵—). In this manner, a compound having a group corresponding to R¹—R²— (=group corresponding to R⁶—R⁵—) at both ends of the perfluoropolyether chain corresponding to R³ is obtained.

As the epoxy compound having a group as R¹—R²— in Formula (1) (=group as =R⁶—R⁵—), for example, a compound represented by Formulae (7-1) to (7-8) can be used.

In a case where the fluorine-based compound and the epoxy compound react with each other, the hydroxy group of the epoxy compound may be protected with an appropriate protective group, and then the fluorine-based compound may react with the epoxy compound.

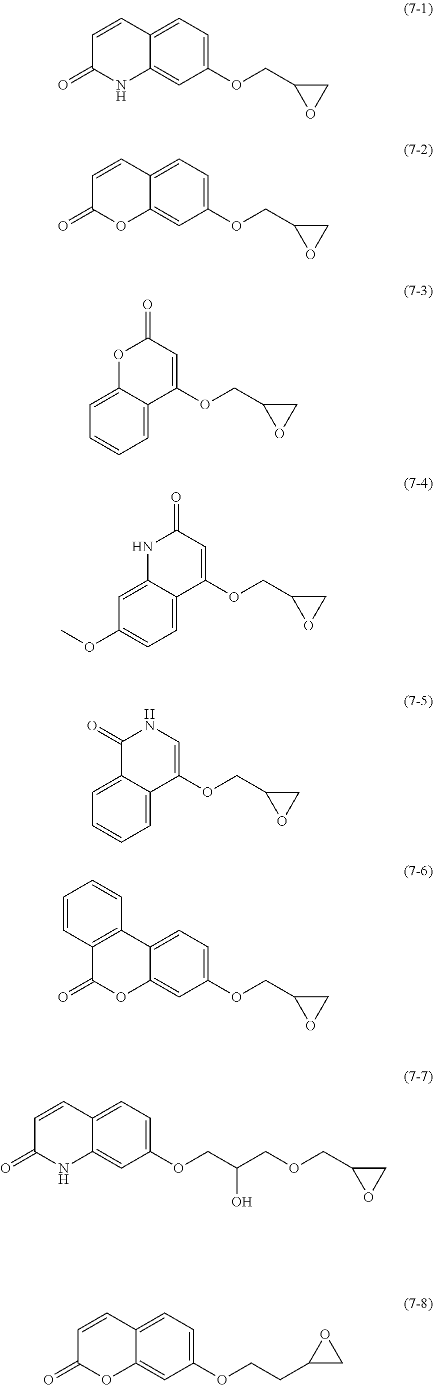

The epoxy compound having a group as R¹—R²— in Formula (1) (that is, a group as R⁶—R⁵—) can be produced, for example, by a method of reacting an alcohol having a structure corresponding to the end group represented by R¹ or R⁶ in Formula (1) (R in Formula (8)) with a halogen compound having an epoxy group corresponding to R² or R⁵, as shown in Formula (8).

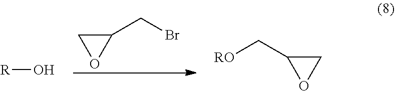

(In Formula (8), R represents a structure corresponding to the end group represented by R¹ or R⁶ in General Formula (1).)

In addition, the above-described epoxy compound may be produced, for example, by a method of performing an addition reaction of an alcohol having a structure corresponding to the end group represented by R¹ or R⁶ in Formula (1) (R in Formula (9)) and allylglycidyl ether and oxidizing the compound obtained by the addition reaction through the action of m-chloroperbenzoic acid (mCPBA), as shown in Formula (9).

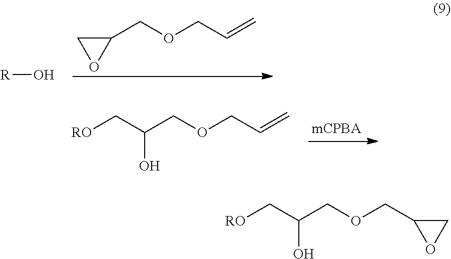

(In Formula (9), R represents a structure corresponding to the end group represented by R¹ or R⁶ in General Formula (1).)

In addition, the above-described epoxy compound may be produced, for example, by a method of reacting an alcohol having a structure corresponding to the end group represented by R¹ or R⁶ in Formula (1) (R in Formula (10)) with a halogen compound having an alkenyl group corresponding to R² or R⁵ as shown in Formula (10) and oxidizing the obtained compound through the action of m-chloroperbenzoic acid (mCPBA).

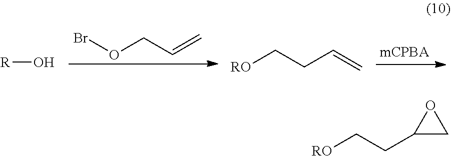

(In Formula (10), R represents a structure corresponding to the end group represented by R¹ or R⁶ in General Formula (1).)

A commercially available product may be purchased and used as the above-described epoxy compound.

By performing the above-described step, a compound in which n in Formula (1) represents 0 and R¹—R²— and R⁶—R⁵— are the same as each other is obtained.

[1-2] in Case where R¹—R²— and R⁶—R⁵— are Different from Each Other

First, a fluorine-based compound in which a hydroxymethyl group (—CH₂OH) is disposed at both ends of a perfluoropolyether chain corresponding to R³ in Formula (1) is prepared.

Next, the hydroxy group of the hydroxymethyl group disposed at one end of the fluorine-based compound reacts with the epoxy group of the epoxy compound having a group represented by R¹—R²— in Formula (1). In this manner, an intermediate compound 1 having a group corresponding to R¹—R²— at one end of the perfluoropolyether chain corresponding to R³ is obtained (first reaction).

The epoxy compound having a group as R¹—R²— may react with the above-described fluorine-based compound after the hydroxy group is protected with an appropriate protective group.

Next, the above-described intermediate compound 1 reacts with the epoxy compound having a group as R⁶—R⁵— in Formula (1) (second reaction).

By performing the above-described step, a compound in which n in Formula (1) represents 0 and R¹—R²— and R⁶—R⁵— are different from each other is obtained.

[2] Production Method in which n Represents 1 in Formula (1)
[2-1] in Case where Two PFPE Chains Represented by R³ are the same as each other and R¹—R²— and R⁶—R⁵— are the Same as Each Other

First, an intermediate compound 1 having a group corresponding to R¹—R²— (that is, a group also corresponding to R⁶—R⁵—) at one end of the perfluoropolyether chain corresponding to R³ is produced in the same manner as in the above-described production method [1-2](first reaction).

Thereafter, the intermediate compound 1 reacts with the halogen compound or the diepoxy compound having an epoxy group corresponding to R⁴ (second reaction). Examples of the halogen compound having an epoxy group corresponding to R⁴ include epibromohydrin, epichlorohydrin, chloroethyloxirane, and bromoethyloxirane. Examples of the diepoxy compound corresponding to R⁴ include diglycidyl ether.

By performing the above-described step, a compound in which n in Formula (1) represents 1, two PFPE chains represented by R³ are the same as each other, and R¹—R²— and R⁶—R⁵— are the same as each other is obtained.

[2-2] in Case where any One or More of Two PFPE Chains Represented by R¹ and R⁶, R² and R⁵, and R³ are Different from Each Other

First, an intermediate compound 1a having a group corresponding to R¹—R²— at one end of the perfluoropolyether chain corresponding to R³ on the R¹ side is produced in the same manner as in the above-described production method [1-2](first reaction).

Next, the intermediate compound 1a reacts with the halogen compound or the diepoxy compound having an epoxy group corresponding to R⁴ to produce an intermediate compound 2 having a group corresponding to R¹—R²— at one end of the perfluoropolyether chain corresponding to R³ on the R¹ side and having an epoxy group at the other end (second reaction).

Next, an intermediate compound 1b having a group corresponding to R⁶—R⁵— at one end of the perfluoropolyether chain corresponding to R³ on the R⁶ side is produced in the same manner as in the first reaction (third reaction). Thereafter, the intermediate compound 2 reacts with the intermediate compound 1b (fourth reaction).

In the above-described step, the compound may be produced by a method of reacting the intermediate compound 1a with a halogen compound having an alkenyl group corresponding to R⁴, oxidizing the obtained compound to obtain an epoxy compound, and reacting the epoxy compound with the intermediate compound 1b. Examples of the halogen compound having an alkenyl group corresponding to R⁴ include allyl chloride, allyl bromide, and 1-bromo-3-butene.

By performing the above-described steps, a compound in which n in Formula (1) represents 1 and any one or more of two PFPE chains represented by R¹ and R⁶, R² and R⁵, or R³ are different from each other can be produced.

[3] Production Method in Case where n in Formula (1) Represents 2
[3-1] in Case where Three PFPE Chains Represented by R³ are the same as each other, R¹—R²— and R⁶—R⁵— are the same as each other, and two linking groups represented by R⁴ are the Same as Each Other

First, a fluorine-based compound in which hydroxymethyl groups (—CH₂OH) are each disposed at both ends of a perfluoropolyether chain corresponding to R³ at the center of the molecule in Formula (1) is prepared.

Next, a hydroxy group of a hydroxymethyl group, which is disposed at both ends of the fluorine-based compound reacts with a halogen compound having an epoxy group corresponding to R⁴ (first reaction). In this manner, an intermediate compound 3 having an epoxy group at both ends of the perfluoropolyether chain corresponding to R³ at the center of the molecule in Formula (1) is obtained.

Next, an intermediate compound 1 having a group corresponding to R¹—R²— (that is, also corresponding to R⁶—R⁵—) at one end of the perfluoropolyether chain corresponding to R³ on the R¹ side (that is, R³ on the R⁶ side) is produced by the same method as the above-described production method [1-2](second reaction).

Thereafter, a hydroxy group of the hydroxymethyl group disposed at one end of the intermediate compound 1 reacts with an epoxy group disposed at both ends of the intermediate compound 3 (third reaction).

By performing the above-described step, a compound in which n in Formula (1) represents 2, three PFPE chains represented by R³ are the same as each other, R¹—R²— and R⁶—R⁵— are the same as each other, and two linking groups represented by R⁴ are the same as each other can be produced. Here, the order of the first reaction and the second reaction may be reversed.

[3-2] in Case where Two Linking Groups Represented by R⁴ are the same as each other, and any one or more of three PFPE chains represented by R¹ and R⁶, R² and R⁵, and R³ are Different from Each Other

First, an intermediate compound 3 is obtained in the same manner as in the first reaction of the production method [3-1]. Next, in the second reaction of the production method [3-1] described above, an intermediate compound 1a having a group corresponding to R¹—R²— at one end of the perfluoropolyether chain corresponding to R³ on the R¹ side and an intermediate compound 1b having a group corresponding to R⁶—R⁵— at one end of the perfluoropolyether chain corresponding to R³ on the R⁶ side are each synthesized. Further, in the third reaction of the production method [3-1] described above, the intermediate compound 1a and the intermediate compound 1b sequentially react with the epoxy groups disposed at each end of the intermediate compound 3.

By performing the above-described steps, a compound in which n in Formula (1) represents 2, two linking groups represented by R⁴ are the same as each other, and any one or more of the three PFPE chains represented by R¹ and R⁶, R² and R⁵, and R³ are different from each other can be produced.

[3-3] in Case where Three PFPE Chains Represented by R³ are the same as each other, R¹—R²— and R⁶—R⁵— are the same as each other, and two linking groups represented by R⁴ are Different from Each Other

In the first reaction of the production method [3-1] described above, two kinds of halogen compounds having an epoxy group, which correspond to the two linking groups represented by R⁴, sequentially react with the hydroxy group of the hydroxymethyl group disposed at each end of the fluorine-based compound, to obtain an intermediate compound 4. Thereafter, the reaction is carried out in the same manner as in the above-described production method [3-1], except that the epoxy group disposed at both ends of the intermediate compound 4 reacts with the intermediate compound 1 in the third reaction of the production method [3-1].

By performing the above-described step, a compound in which n in Formula (1) represents 2, three PFPE chains represented by R³ are the same as each other, R¹—R²— and R⁶—R⁵— are the same as each other, and two linking groups represented by R⁴ are different from each other can be produced.

The fluorine-containing ether compound of the present embodiment is a compound represented by Formula (1), in which a divalent linking group R² or R⁵ having one or more polar groups and an end group R¹ or R⁶ are bonded in this order to both sides of a skeleton (—CH₂—R³[—CH₂—R⁴—CH₂—R³]ₙ—CH₂—) having a PFPE chain represented by R³, and at least one of the end groups R¹ or R⁶ is a fluorine-containing ether compound which is a condensed aromatic group having an amide or an ester represented by Formula (2). Therefore, the lubricating layer formed on the protective layer using the lubricant containing the fluorine-containing ether compound according to the present embodiment has satisfactory chemical substance resistance and suppressed pickup.

### [Lubricant for Magnetic Recording Medium]

A lubricant for a magnetic recording medium according to the present embodiment contains the fluorine-containing ether compound represented by Formula (1).

In the lubricant of the present embodiment, as long as the characteristics are not impaired by containing the fluorine-containing ether compound represented by Formula (1), known materials used as a material for the lubricant can be used in the form of a mixture as necessary.

Specific examples of the known materials include, for example, FOMBLIN (registered trademark) ZDIAC, FOMBLIN ZDEAL, and FOMBLIN AM-2001 (all manufactured by Solvay Solexis), and Moresco A20H (manufactured by Moresco). The known materials used in the form of a mixture with the lubricant according to the present embodiment has a number-average molecular weight of preferably 1000 to 10000.

In a case where the lubricant of the present embodiment contains a material other than the fluorine-containing ether compound represented by Formula (1), the content of the fluorine-containing ether compound represented by Formula (1) in the lubricant of the present embodiment is preferably 50% by mass or greater and more preferably 70% by mass or greater.

Since the lubricant of the present embodiment contains the fluorine-containing ether compound represented by Formula (1), a lubricating layer having excellent chemical substance resistance and excellent pickup suppression can be formed.

### [Magnetic Recording Medium]

The magnetic recording medium according to the present embodiment is formed by sequentially providing at least a magnetic layer, a protective layer, and a lubricating layer on a substrate.

In the magnetic recording medium according to the present embodiment, one or two or more underlayers can be provided between the substrate and the magnetic layer as necessary. In addition, at least one of an adhesive layer or a soft magnetic layer can be provided between the underlayer and the substrate.

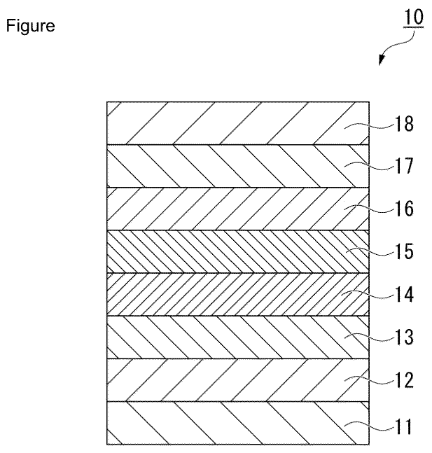

FIG. 1 is a schematic cross-sectional view showing an embodiment of the magnetic recording medium according to the present invention.

The magnetic recording medium 10 according to the present embodiment has a structure in which an adhesive layer 12, a soft magnetic layer 13, a first underlayer 14, a second underlayer 15, a magnetic layer 16, a protective layer 17, and a lubricating layer 18 are sequentially provided on a substrate 11.

### “Substrate”

As the substrate 11, for example, a non-magnetic substrate in which a film made of NiP or a NiP alloy is formed on a base made of a metal or an alloy material such as Al or an Al alloy can be used.

In addition, as the substrate 11, a non-magnetic substrate made of a non-metal material such as glass, ceramics, silicon, silicon carbide, carbon, or a resin may be used, or a non-magnetic substrate in which a film of NiP or a NiP alloy is formed on a base made of these non-metal materials may be used.

The glass substrate has rigidity and excellent smoothness, and is suitable for increasing the recording density. Examples of the glass substrate include an aluminosilicate glass substrate, and a chemically strengthened aluminosilicate glass substrate is particularly suitable.

it is preferable that the roughness of the main surface of the substrate 11 has a Rmax of 6 nm or less and a Ra of 0.6 nm or less so that the main surface thereof is ultrasmooth. Further, the surface roughness Rmax and the surface roughness Ra referred to herein are based on the regulations of JIS B 0601.

### “Adhesive Layer”

The adhesive layer 12 prevents the progression of corrosion of the substrate 11 in a case where the substrate 11 and the soft magnetic layer 13 provided on the adhesive layer 12 are disposed in contact with each other.

The material of the adhesive layer 12 can be appropriately selected from, for example, Cr, a Cr alloy, Ti, a Ti alloy, CrTi, NiAl, and an AlRu alloy. The adhesive layer 12 can be formed by, for example, a sputtering method.

### “Soft Magnetic Layer”

It is preferable that the soft magnetic layer 13 has a structure in which a first soft magnetic film, an intermediate layer formed of a Ru film, and a second soft magnetic film are sequentially laminated. That is, it is preferable that the soft magnetic layer 13 has a structure in which the upper and lower soft magnetic films of the intermediate layer are antiferro-coupled (AFC) by interposing the intermediate layer formed of a Ru film between the two layers of the soft magnetic films.

Examples of the material of the first soft magnetic film and the second soft magnetic film include a CoZrTa alloy and a CoFe alloy.

It is preferable to add any of Zr, Ta, or Nb to the CoFe alloy used in the first soft magnetic film and the second soft magnetic film. In this manner, the amorphization of the first soft magnetic film and the second soft magnetic film is promoted so that the aligning properties of the first underlayer (seed layer) can be improved, and the floating amount of the magnetic head can be reduced.

The soft magnetic layer 13 can be formed by, for example, a sputtering method.

### “First Underlayer”

The first underlayer 14 is a layer for controlling the alignment and the crystal size of the second underlayer 15 and the magnetic layer 16 provided thereon.

Examples of the first underlayer 14 include a Cr layer, a Ta layer, a Ru layer, a CrMo alloy layer, a CoW alloy layer, a CrW alloy layer, a CrV alloy layer, and a CrTi alloy layer.

The first underlayer 14 can be formed by, for example, a sputtering method.

### “Second Underlayer”

The second underlayer 15 is a layer in which the alignment of the magnetic layer 16 is controlled to be satisfactory. The second underlayer 15 is preferably a layer formed of Ru or a Ru alloy.

The second underlayer 15 may consist of a single layer or a plurality of layers. In a case where the second underlayer 15 is formed of a plurality of layers, all the layers may be made of the same material, or at least one layer may be made of a different material.

The second underlayer 15 can be formed by, for example, a sputtering method.

### “Magnetic Layer”

The magnetic layer 16 is formed of a magnetic film in which a magnetization easy axis is oriented in a direction perpendicular or horizontal to the substrate surface. The magnetic layer 16 is a layer containing Co and Pt, and may be a layer containing an oxide, Cr, B, Cu, Ta, Zr, or the like in order to further improve the SNR characteristics.

Examples of the oxide contained in the magnetic layer 16 include SiO₂, SiO, Cr₂O₃, CoO, Ta₂O₃, and TiO₂.

The magnetic layer 16 may consist of one layer or a plurality of magnetic layers formed of materials having different compositions.

For example, in a case where the magnetic layer 16 consists of three layers of a first magnetic layer, a second magnetic layer, and a third magnetic layer which are laminated in this order from the bottom, it is preferable that the first magnetic layer has a granular structure formed of a material containing Co, Cr, and Pt and further containing an oxide. As the oxide contained in the first magnetic layer, for example, an oxide of Cr, Si, Ta, Al, Ti, Mg, or Co is preferably used. Among these, TiO₂, Cr₂O₃, SiO₂, or the like can be particularly suitably used. In addition, it is preferable that the first magnetic layer is formed of a composite oxide to which two or more kinds of oxides are added. Among these, Cr₂O₃—SiO₂, Cr₂O₃—TiO₂, SiO₂—TiO₂, or the like can be particularly suitably used.

The first magnetic layer can contain at least one element selected from the group consisting of B, Ta, Mo, Cu, Nd, W, Nb, Sm, Tb, Ru, and Re, in addition to Co, Cr, Pt, and an oxide.

The same material as that of the first magnetic layer can be used for the second magnetic layer. It is preferable that the second magnetic layer has a granular structure.

It is preferable that the third magnetic layer has a non-granular structure formed of a material containing Co, Cr, and Pt and not containing an oxide. The third magnetic layer can contain at least one element selected from the group consisting of B, Ta, Mo, Cu, Nd, W, Nb, Sm, Tb, Ru, Re, and Mn, in addition to Co, Cr, and Pt.

In a case where the magnetic layer 16 is formed of a plurality of magnetic layers, it is preferable to provide a non-magnetic layer between the adjacent magnetic layers. In a case where the magnetic layer 16 is formed of three layers of a first magnetic layer, a second magnetic layer, and a third magnetic layer, it is preferable to provide a non-magnetic layer between the first magnetic layer and the second magnetic layer and between the second magnetic layer and the third magnetic layer.

As the non-magnetic layer provided between the magnetic layers 16 adjacent to each other, for example, Ru, a Ru alloy, a CoCr alloy, or a CoCrX1 alloy (X1 represents at least one element selected from the group consisting of Pt, Ta, Zr, Re, Ru, Cu, Nb, Ni, Mn, Ge, Si, O, N, W, Mo, Ti, V, Zr, and B) can be suitably used.

It is preferable that, as the non-magnetic layer provided between adjacent magnetic layers of the magnetic layer 16, an alloy material containing an oxide, a metal nitride, or a metal carbide is used. Specifically, for example, SiO₂, Al₂O₃, Ta₂O₅, Cr₂O₃, MgO, Y₂O₃, or TiO₂ can be used as the oxide. As the metal nitride, for example, AlN, Si₃N₄, TaN, or CrN can be used. As the metal carbide, for example, TaC, BC, or SiC can be used.

The non-magnetic layer can be formed by, for example, a sputtering method.

In order to achieve a higher recording density, it is preferable that the magnetic layer 16 is a magnetic layer for perpendicular magnetic recording in which a magnetization easy axis is oriented in a direction perpendicular to the substrate surface. The magnetic layer 16 may be a magnetic layer for in-plane magnetic recording.

The magnetic layer 16 may be formed by any of the known methods of the related art, such as a evaporation method, an ion beam sputtering method, and a magnetron sputtering method. The magnetic layer 16 is usually formed by a sputtering method.

### “Protective Layer”

The protective layer 17 protects the magnetic layer 16. The protective layer 17 may be formed of one layer or a plurality of layers. As the protective layer 17, a carbon-based protective layer can be preferably used, and an amorphous carbon protective layer is particularly preferable. In a case where the protective layer 17 is a carbon-based protective layer, the interaction with the polar group (particularly, the hydroxy group) contained in the fluorine-containing ether compound in the lubricating layer 18 is further enhanced, which is preferable.

The adhesion force between the carbon-based protective layer and the lubricating layer 18 can be controlled by forming the carbon-based protective layer with hydrocarbon and/or nitrogenated carbon and adjusting the hydrogen content and/or the nitrogen content in the carbon-based protective layer. The hydrogen content in the carbon-based protective layer is preferably in a range of 3% by atom to 20% by atom in a case of being measured by a hydrogen forward scattering method (HFS). In addition, the nitrogen content in the carbon-based protective layer is preferably in a range of 4% by atom to 15% by atom in a case where the nitrogen content is measured by X-ray photoelectron spectroscopy (XPS).

The hydrogen and/or nitrogen contained in the carbon-based protective layer are not necessarily uniformly contained in the entire carbon-based protective layer. It is suitable that the carbon-based protective layer is, for example, a composition gradient layer in which nitrogen is contained on the lubricating layer 18 side of the protective layer 17 and hydrogen is contained on the magnetic layer 16 side of the protective layer 17. In this case, the adhesion force between the magnetic layer 16, the lubricating layer 18, and the carbon-based protective layer is further improved.

The film thickness of the protective layer 17 is preferably in a range of 1 nm to 7 nm. In a case where the film thickness of the protective layer 17 is 1 nm or greater, the performance as the protective layer 17 can be sufficiently obtained. It is preferable that the film thickness of the protective layer 17 is 7 nm or less from the viewpoint of reducing the film thickness of the protective layer 17.

As a film forming method of the protective layer 17, a sputtering method using a target material containing carbon, a chemical vapor deposition (CVD) method using a hydrocarbon raw material such as ethylene or toluene, an ion beam deposition (IBD) method, or the like can be used.

In a case where a carbon-based protective layer is formed as the protective layer 17, the carbon-based protective layer can be formed, for example, by a DC magnetron sputtering method. In particular, in a case where a carbon-based protective layer is formed as the protective layer 17, it is preferable to form an amorphous carbon protective layer by a plasma CVD method. The amorphous carbon protective layer formed by the plasma CVD method has a uniform surface and small roughness.

### “Lubricating Layer”

The lubricating layer 18 prevents contamination of the magnetic recording medium 10. In addition, the lubricating layer 18 reduces a frictional force of the magnetic head of the magnetic recording and reproducing device that slides on the magnetic recording medium 10 to improve the durability of the magnetic recording medium 10.

As shown in FIG. 1, the lubricating layer 18 is formed in contact with the protective layer 17. The lubricating layer 18 is formed by applying the lubricant for a magnetic recording medium according to the embodiment described above onto the protective layer 17. Therefore, the lubricating layer 18 contains the above-described fluorine-containing ether compound.

In a case where the protective layer 17 disposed below the lubricating layer 18 is a carbon-based protective layer, the lubricating layer 18 is particularly bonded to the protective layer 17 with a high binding force. As a result, even in a case where the thickness of the lubricating layer 18 is small, the magnetic recording medium 10 in which the surface of the protective layer 17 is coated at a high coating rate is likely to be obtained, and the contamination of the surface of the magnetic recording medium 10 can be effectively prevented.

The average film thickness of the lubricating layer 18 is preferably in a range of 0.5 nm (5 Å) to 2.0 nm (20 Å) and more preferably in a range of 0.5 nm (5 Å) to 1.2 nm (12 Å). In a case where the average film thickness of the lubricating layer 18 is 0.5 nm or greater, the lubricating layer 18 is formed with a uniform film thickness without being island-like or mesh-like. Therefore, the surface of the protective layer 17 can be coated with the lubricating layer 18 at a high coating rate. In addition, in a case where the average film thickness of the lubricating layer 18 is 2.0 nm or less, the lubricating layer 18 can be sufficiently thinned, and the floating amount of the magnetic head can be sufficiently reduced.

### “Method for Forming Lubricating Layer”

Examples of a method of forming the lubricating layer 18 include a method of preparing a magnetic recording medium in the middle of production in which each layer up to the protective layer 17 is formed on the substrate 11 and coating the protective layer 17 with a lubricating layer forming solution.

The lubricating layer forming solution is obtained by dispersing and dissolving the lubricant for a magnetic recording medium according to the embodiment described above in a solvent as necessary, and setting the viscosity and the concentration suitable for a coating method.

Examples of a solvent used in the lubricating layer forming solution include a fluorine-based solvent such as VERTREL (registered trademark) XF (trade name, manufactured by DuPont Mitsui Fluorochemicals Co., Ltd.) or ASAHIKLIN (registered trademark) AE-3000 (trade name, manufactured by AGC Inc.).

A coating method for the lubricating layer forming solution is not particularly limited, and examples thereof include a spin coating method, a spraying method, a paper coating method, and a dipping method.

In a case where the dipping method is used, for example, the following method can be used. First, the substrate 11 on which each layer up to the protective layer 17 is formed is immersed in a lubricating layer forming solution placed in an immersion tank of a dip coating device. Next, the substrate 11 is pulled up from the immersion tank at a predetermined speed. In this manner, the lubricating layer forming solution is applied onto the surface of the protective layer 17 of the substrate 11.

By using the dipping method, the lubricating layer forming solution can be uniformly applied to the surface of the protective layer 17, and the lubricating layer 18 can be formed on the protective layer 17 with a uniform film thickness.

In the present embodiment, it is preferable to perform a thermal treatment on the substrate 11 on which the lubricating layer 18 has been formed. By performing the thermal treatment, the adhesion between the lubricating layer 18 and the protective layer 17 is improved, and the adhesion force between the lubricating layer 18 and the protective layer 17 is improved.

The thermal treatment temperature is preferably in a range of 100° C. to 180° C. and more preferably in a range of 100° C. to 160° C. In a case where the thermal treatment temperature is 100° C. or higher, the effect of improving the adhesion between the lubricating layer 18 and the protective layer 17 is sufficiently obtained. In addition, thermal decomposition of the lubricating layer 18 can be prevented by setting the thermal treatment temperature to 180° C. or lower. The thermal treatment time can be appropriately adjusted according to the thermal treatment temperature and is preferably 10 minutes to 120 minutes.

In the present embodiment, in order to further improve the adhesion force of the lubricating layer 18 to the protective layer 17, a treatment of irradiating the lubricating layer 18 with ultraviolet rays (UV) may be performed before or after the thermal treatment.

The magnetic recording medium 10 according to the present embodiment is formed by sequentially providing at least the magnetic layer 16, the protective layer 17, and the lubricating layer 18 on the substrate 11. In the magnetic recording medium 10 according to the present embodiment, the lubricating layer 18 containing the above-described fluorine-containing ether compound is formed in contact with the protective layer 17. The lubricating layer 18 has excellent adhesion even in a case where the film thickness thereof is small, and has satisfactory chemical substance resistance and a satisfactory pickup suppression effect. Therefore, the magnetic recording medium 10 according to the present embodiment has excellent reliability, suppresses particularly silicon contamination, and has excellent durability. Therefore, the magnetic recording medium 10 according to the present embodiment has a low magnetic head floating amount (for example, 10 nm or less), and stably operates for a long period of time even in a severe environment due to the diversification of applications. Therefore, the magnetic recording medium 10 according to the present embodiment is particularly suitable as a magnetic disk mounted on a magnetic disk apparatus of a load unload (LUL) system.

### EXAMPLES

Hereinafter, the present invention will be described in more detail with reference to examples and comparative examples. Further, the present invention is not limited to the following examples.

### Example 1

A compound represented by Formula (A) was obtained by the following method.

A 100 mL eggplant flask was charged with 20 g of a compound represented by HOCH₂CF₂O(CF₂CF₂O)ₕ(CF₂O)ᵢCF₂CH₂OH (h representing the average degree of polymerization in the formula represent 4.5, and i representing the average degree of polymerization represent 4.5) (number-average molecular weight: 1,000, molecular weight distribution: 1.1), 4.34 g of a compound represented by Formula (7-1), and 20 mL of t-butanol in a nitrogen gas atmosphere, and the mixture was stirred at room temperature until the mixture was uniform. 1.12 g of potassium tert-butoxide was added to the mixture, and the mixture was stirred at 70° C. for 16 hours to carry out a reaction (first reaction).

Further, the compound represented by Formula (7-1) was synthesized by reacting 7-hydroxyquinolinone with epibromohydrin.

The reaction product obtained after the reaction was cooled to room temperature, transferred to a separatory funnel containing 100 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 9.73 g of a compound represented by Formula (11) as an intermediate compound.

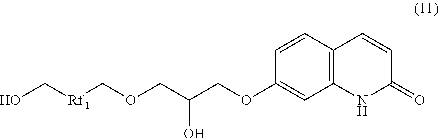

(In Rf₁ in Formula (11), h representing the average degree of polymerization represents 4.5, and i representing the average degree of polymerization represents 4.5.)

Subsequently, a 100 mL eggplant flask was charged with 9.74 g of the compound represented by Formula (11) as an intermediate compound obtained above, 1.62 g of a compound represented by Formula (12), and 10 mL of t-butanol in a nitrogen gas atmosphere, and the mixture was stirred at room temperature until the mixture was uniform. 0.45 g of potassium tert-butoxide was added to the uniform liquid, and the mixture was stirred at 70° C. for 23 hours to carry out a reaction (second reaction).

Further, the compound represented by Formula (12) was synthesized by oxidizing a compound obtained by protecting ethylene glycol monoallyl ether with dihydropyran.

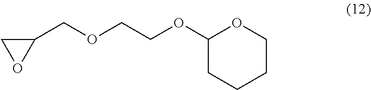 [SMILES: C1CCC(OCCOCC2CO2)OC1]

The reaction solution obtained after the reaction was cooled to room temperature, 18 g of a 10% hydrogen chloride/methanol solution (hydrogen chloride-methanol reagent (5-10%), manufactured by Tokyo Chemical Industry Co., Ltd.) was added thereto, and the mixture was stirred at room temperature for 2 hours. The reaction solution was transferred to a separatory funnel containing 100 mL of water, and was extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 5.11 g of a compound (A) (in Rf₁ in Formula (A), h representing the average degree of polymerization represents 4.5 and i representing the average degree of polymerization represents 4.5).

The structure of the obtained compound (A) was identified by performing ¹H-NMR measurement and ¹⁹F-NMR measurement based on the following results.

¹H-NMR (acetone-D₆): δ [ppm]=3.40-4.00 (17H), 4.10-4.20 (4H), 6.30-7.80 (6H)

¹⁹F-NMR (acetone-D₆): δ [ppm]=−55.5 to −51.5 (9F), −78.5 (2F), −80.5 (2F), −91.0 to −88.5 (18F)

### Example 2

A compound represented by Formula (B) was obtained by the following method.

4.89 g of a compound (B) (in Rf₁ in Formula (B), h representing the average degree of polymerization represents 4.5 and i representing the average degree of polymerization represents 4.5) was obtained by performing the same operation as in Example 1 except that a compound represented by Formula (7-2) was used instead of the compound represented by Formula (7-1) in Example 1.

Further, the compound represented by Formula (7-2) was synthesized by reacting 7-hydroxycoumarin with epibromohydrin.

The structure of the obtained compound (B) was identified by performing ¹H-NMR measurement and ¹⁹F-NMR measurement based on the following results.

¹H-NMR (acetone-D₆): δ [ppm]=3.40-4.00 (17H), 4.10-4.20 (4H), 6.30-7.80 (5H)

¹⁹F-NMR (acetone-D₆): δ [ppm]=−55.5 to −51.5 (9F), −78.5 (2F), −80.5 (2F), −91.0 to −88.5 (18F)

### Example 3

A compound represented by Formula (C) was obtained by the following method.

4.96 g of a compound (C) (in Rf₁ in Formula (C), h representing the average degree of polymerization represents 4.5 and i representing the average degree of polymerization represents 4.5) was obtained by performing the same operation as in Example 1 except that a compound represented by Formula (7-3) was used instead of the compound represented by Formula (7-1) in Example 1.

Further, the compound represented by Formula (7-3) was synthesized by reacting 4-hydroxycoumarin with epibromohydrin.

The structure of the obtained compound (C) was identified by performing ¹H-NMR measurement and ¹⁹F-NMR measurement based on the following results.

¹H-NMR (acetone-D₆): δ [ppm]=3.40-4.00 (17H), 4.10-4.20 (4H), 6.30-7.80 (5H)

¹⁹F-NMR (acetone-D₆): δ [ppm]=−55.5 to −51.5 (9F), −78.5 (2F), −80.5 (2F), −91.0 to −88.5 (18F)

### Example 4

A compound represented by Formula (D) was obtained by the following method.

5.23 g of a compound (D) (in Rf₁ in Formula (D), h representing the average degree of polymerization represents 4.5 and i representing the average degree of polymerization represents 4.5) was obtained by performing the same operation as in Example 1 except that a compound represented by Formula (7-4) was used instead of the compound represented by Formula (7-1) in Example 1.

Further, the compound represented by Formula (7-4) was synthesized by reacting 4-hydroxy-7-methoxyquinolinone with epibromohydrin.

The structure of the obtained compound (D) was identified by performing ¹H-NMR measurement and ¹⁹F-NMR measurement based on the following results.

¹H-NMR (acetone-D₆): δ [ppm]=3.40-4.00 (20H), 4.10-4.20 (4H), 6.30-7.80 (5H)

¹⁹F-NMR (acetone-D₆): δ [ppm]=−55.5 to −51.5 (9F), −78.5 (2F), −80.5 (2F), −91.0 to −88.5 (18F)

### Example 5

A compound represented by Formula (E) was obtained by the following method.

5.03 g of a compound (E) (in Rf₁ in Formula (E), h representing the average degree of polymerization represents 4.5 and i representing the average degree of polymerization represents 4.5) was obtained by performing the same operation as in Example 1 except that a compound represented by Formula (7-5) was used instead of the compound represented by Formula (7-1) in Example 1.

Further, the compound represented by Formula (7-5) was synthesized by reacting 4-hydroxy-2H-isoquinolin-1-one with epibromohydrin.

The structure of the obtained compound (E) was identified by performing ¹H-NMR measurement and ¹⁹F-NMR measurement based on the following results.

¹H-NMR (acetone-D₆): δ [ppm]=3.40-4.00 (17H), 4.10-4.20 (4H), 6.30-7.80 (6H)

¹⁹F-NMR (acetone-D₆): δ [ppm]=−55.5 to −51.5 (9F), −78.5 (2F), −80.5 (2F), −91.0 to −88.5 (18F)

### Example 6

A compound represented by Formula (F) was obtained by the following method.

5.28 g of a compound (F) (in Rf₁ in Formula (F), h representing the average degree of polymerization represents 4.5 and i representing the average degree of polymerization represents 4.5) was obtained by performing the same operation as in Example 1 except that a compound represented by Formula (7-6) was used instead of the compound represented by Formula (7-1) in Example 1.

Further, the compound represented by Formula (7-6) was synthesized by reacting 3-hydroxy-6H-benzo[c]chromen-6-one with epibromohydrin.

The structure of the obtained compound (F) was identified by performing ¹H-NMR measurement and ¹⁹F-NMR measurement based on the following results.

¹H-NMR (acetone-D₆): δ [ppm]=3.40-4.00 (17H), 4.10-4.20 (4H), 6.30-7.80 (8H)

¹⁹F-NMR (acetone-D₆): δ [ppm]=−55.5 to −51.5 (9F), −78.5 (2F), −80.5 (2F), −91.0 to −88.5 (18F)

### Example 7

A compound represented by Formula (G) was obtained by the following method.

A compound (G) produced as a by-product in the first reaction of Example 1 was purified by silica gel column chromatography, thereby obtaining 4.22 g of a compound (G) (in Rf₁ in Formula (G), h representing the average degree of polymerization represents 4.5 and i representing the average degree of polymerization represents 4.5).

The structure of the obtained compound (G) was identified by performing ¹H-NMR measurement and ¹⁹F-NMR measurement based on the following results.

¹H-NMR (acetone-D₆): δ [ppm]=3.40-4.00 (12H), 4.10-4.20 (4H), 6.30-7.80 (12H)

¹⁹F-NMR (acetone-D₆): δ [ppm]=−55.5 to −51.5 (9F), −78.5 (2F), −80.5 (2F), −91.0 to −88.5 (18F)

### Example 8

A compound represented by Formula (H) was obtained by the following method.

A compound (H) produced as a by-product in the first reaction of Example 2 was purified by silica gel column chromatography, thereby obtaining 4.05 g of a compound (H) (in Rf₁ in Formula (H), h representing the average degree of polymerization represents 4.5 and i representing the average degree of polymerization represents 4.5).

The structure of the obtained compound (H) was identified by performing ¹H-NMR measurement and ¹⁹F-NMR measurement based on the following results.

¹H-NMR (acetone-D₆): δ [ppm]=3.40-4.00 (12H), 4.10-4.20 (4H), 6.30-7.80 (10H)

¹⁹F-NMR (acetone-D₆): δ [ppm]=−55.5 to −51.5 (9F), −78.5 (2F), −80.5 (2F), −91.0 to −88.5 (18F)

### Example 9

A compound represented by Formula (I) was obtained by the following method.

4.52 g of a compound (I) (in Rf₁ in Formula (I), h representing the average degree of polymerization represents 4.5 and i representing the average degree of polymerization represents 4.5) was obtained by performing the same operation as in Example 1 except that a compound represented by Formula (13) was used instead of the compound represented by Formula (12) in Example 1.

Further, the compound represented by Formula (13) was synthesized by reacting allyl alcohol with allyl glycidyl ether and oxidizing one double bond through the action of m-chloroperbenzoic acid.

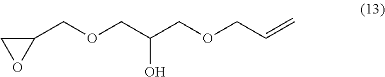

The structure of the obtained compound (I) was identified by performing ¹H-NMR measurement and ¹⁹F-NMR measurement based on the following results.

¹H-NMR (acetone-D₆): δ [ppm]=3.40-4.00 (20H), 4.10-4.20 (4H), 5.20-6.00 (3H), 6.30-7.80 (6H)

¹⁹F-NMR (acetone-D₆): δ [ppm]=−55.5 to −51.5 (9F), −78.5 (2F), −80.5 (2F), −91.0 to −88.5 (18F)

### Example 10

A compound represented by Formula (J) was obtained by the following method.

4.65 g of a compound (J) (in Rf₁ in Formula (J), h representing the average degree of polymerization represents 4.5 and i representing the average degree of polymerization represents 4.5) was obtained by performing the same operation as in Example 2 except that a compound represented by Formula (14) was used instead of the compound represented by Formula (12) in Example 2.

Further, the compound represented by Formula (14) was synthesized by reacting phenol with epibromohydrin.

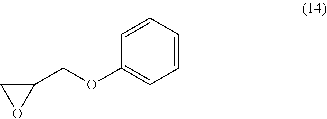 [SMILES: c1ccc(OCC2CO2)cc1]

The structure of the obtained compound (J) was identified by performing ¹H-NMR measurement and ¹⁹F-NMR measurement based on the following results.

¹H-NMR (acetone-D₆): δ [ppm]=3.40-4.00 (12H), 4.10-4.20 (4H), 6.30-7.80 (10H)

¹⁹F-NMR (acetone-D₆): δ [ppm]=−55.5 to −51.5 (9F), −78.5 (2F), −80.5 (2F), −91.0 to −88.5 (18F)

### Example 11

A compound represented by Formula (K) was obtained by the following method.

4.98 g of a compound (K) (in Rf₁ in Formula (K), h representing the average degree of polymerization represents 4.5 and i representing the average degree of polymerization represents 4.5) was obtained by performing the same operation as in Example 1 except that a compound represented by Formula (7-7) was used instead of the compound represented by Formula (7-1) in Example 1.

Further, the compound represented by Formula (7-7) was synthesized by reacting 7-hydroxyquinolinone with allyl glycidyl ether and acting m-chloroperbenzoic acid.

The structure of the obtained compound (K) was identified by performing ¹H-NMR measurement and ¹⁹F-NMR measurement based on the following results.

¹H-NMR (acetone-D₆): δ [ppm]=3.40-4.00 (23H), 4.10-4.20 (4H), 6.30-7.80 (6H)

¹⁹F-NMR (acetone-D₆): δ [ppm]=−55.5 to −51.5 (9F), −78.5 (2F), −80.5 (2F), −91.0 to −88.5 (18F)

### Example 12

A compound represented by Formula (L) was obtained by the following method.

4.56 g of a compound (L) (in Rf₁ in Formula (L), h representing the average degree of polymerization represents 4.5 and i representing the average degree of polymerization represents 4.5) was obtained by performing the same operation as in Example 1 except that a compound represented by Formula (7-8) was used instead of the compound represented by Formula (7-1) in Example 1.

Further, the compound represented by Formula (7-8) was synthesized by reacting 7-hydroxycoumarin with 4-bromo-1-butene and acting m-chloroperbenzoic acid.

The structure of the obtained compound (L) was identified by performing ¹H-NMR measurement and ¹⁹F-NMR measurement based on the following results.

¹H-NMR (acetone-D₆): δ [ppm]=1.40-1.80 (4H), 3.40-4.00 (17H), 4.10-4.20 (4H), 6.30-7.80 (5H)

¹⁹F-NMR (acetone-D₆): δ [ppm]=−55.5 to −51.5 (9F), −78.5 (2F), −80.5 (2F), −91.0 to −88.5 (18F)

### Example 13

A compound represented by Formula (M) was obtained by the following method.

5.21 g of a compound (L) (in Rf₂ in Formula (L), j representing the average degree of polymerization represents 4.5) was obtained by performing the same operation as in Example 1 except that a compound represented by HOCH₂CF₂CF₂O(CF₂CF₂CF₂O)ⱼCF₂CF₂CH₂OH (j representing the average degree of polymerization in the formula represents 4.5) (number-average molecular weight: 1,000, molecular weight distribution: 1.1) was used instead of the compound represented by HOCH₂CF₂O(CF₂CF₂O)ₕ(CF₂O)ᵢCF₂CH₂OH in Example 1.

The structure of the obtained compound (M) was identified by performing ¹H-NMR measurement and ¹⁹F-NMR measurement based on the following results.

¹H-NMR (acetone-D₆): δ [ppm]=3.40-4.00 (17H), 4.10-4.20 (4H), 6.30-7.80 (6H)

¹⁹F-NMR (acetone-D₆): δ [ppm]=−84.0 to −83.0 (18F), −86.4 (4F), −124.3 (4F), −130.0 to −129.0 (9F)

### Example 14

A compound represented by Formula (N) was obtained by the following method.

5.06 g of a compound (N) (in Rf₃ in Formula (N), k representing the average degree of polymerization represents 3.0) was obtained by performing the same operation as in Example 2 except that a compound represented by HOCH₂CF₂CF₂CF₂O(CF₂CF₂CF₂CF₂O)ₖCF₂CF₂CF₂CH₂OH (k representing the average degree of polymerization in the formula represents 3.0) (number-average molecular weight: 1,000, molecular weight distribution: 1.1) was used instead of the compound represented by HOCH₂CF₂O(CF₂CF₂O)ₕ(CF₂O)ᵢCF₂CH₂OH in Example 2.

The structure of the obtained compound (N) was identified by performing ¹H-NMR measurement and ¹⁹F-NMR measurement based on the following results.

¹H-NMR (acetone-D₆): δ [ppm]=3.40-4.00 (17H), 4.10-4.20 (4H), 6.30-7.80 (5H)

¹⁹F-NMR (acetone-D₆): δ [ppm]=−84.0 to −83.0 (16F), −122.5 (4F), −126.0 (12F), −129.0 to −128.0 (4F)

### Example 15

A compound represented by Formula (O) was obtained by the following method.

First, 9.98 g of an intermediate compound represented by Formula (11) was obtained by the first reaction of Example 1. Subsequently, a 100 mL eggplant flask was charged with 9.98 g of the intermediate compound represented by Formula (11) obtained above, 0.56 g of epibromohydrin, and 10 mL of t-butanol in a nitrogen gas atmosphere, and the mixture was stirred at room temperature until the mixture was uniform. 0.92 g of potassium tert-butoxide was added to the uniform liquid, and the mixture was stirred at 70° C. for 23 hours to carry out a reaction.

The reaction solution obtained after the reaction was cooled to room temperature, transferred to a separatory funnel charged with 100 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 4.62 g of a compound (O) (in two Rf₁'s in Formula (O), h representing the average degree of polymerization represents 4.5 and i representing the average degree of polymerization represents 4.5).

The structure of the obtained compound (O) was identified by performing ¹H-NMR measurement and ¹⁹F-NMR measurement based on the following results.

¹H-NMR (acetone-D₆): δ [ppm]=3.40-4.00 (18H), 4.10-4.20 (8H), 6.30-7.80 (12H)

¹⁹F-NMR (acetone-D₆): δ [ppm]=−55.5 to −51.5 (18F), −78.5 (4F), −80.5 (4F), −91.0 to −88.5 (36F)

### Example 16

A compound represented by Formula (P) was obtained by the following method.

4.52 g of a compound (P) (in two Rf₁'s in Formula (P), h representing the average degree of polymerization represents 4.5 and i representing the average degree of polymerization represents 4.5) was obtained by performing the same operation as in Example 15 except that a compound represented by Formula (7-2) was used instead of the compound represented by Formula (7-1) in Example 15.

The structure of the obtained compound (P) was identified by performing ¹H-NMR measurement and ¹⁹F-NMR measurement based on the following results.

¹H-NMR (acetone-D₆): δ [ppm]=3.40-4.00 (18H), 4.10-4.20 (8H), 6.30-7.80 (10H)

¹⁹F-NMR (acetone-D₆): δ [ppm]=−55.5 to −51.5 (18F), −78.5 (4F), −80.5 (4F), −91.0 to −88.5 (36F)

### Example 17

A compound represented by Formula (Q) was obtained by the following method.

A 200 mL eggplant flask was charged with 12 g of a compound represented by HOCH₂CF₂O(CF₂CF₂O)ₕ(CF₂O)ᵢCF₂CH₂OH (h representing the average degree of polymerization in the formula represents 4.5, and i representing the average degree of polymerization represents 4.5), 2.43 g of a compound represented by Formula (12), and 12 mL of t-butanol in a nitrogen gas atmosphere, and the mixture was stirred at room temperature until the mixture was uniform. 0.67 g of potassium tert-butoxide was further added to the uniform liquid, and the mixture was stirred at 70° C. for 16 hours to carry out a reaction.

The reaction product obtained after the reaction was cooled to 25° C., transferred to a separatory funnel containing 100 mL of water, and extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 5.77 g of an intermediate compound represented by Formula (15).

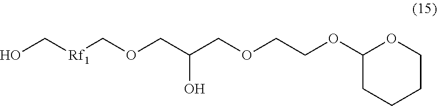

(In Rf₁ in Formula (15), h representing the average degree of polymerization represents 4.5 and i representing the average degree of polymerization represents 4.5.)

Subsequently, a 100 mL eggplant flask was charged with 5.77 g of the intermediate compound represented by Formula (15) obtained above, 6.38 g of a compound represented by Formula (16), and 10 mL of t-butanol in a nitrogen gas atmosphere, and the mixture was stirred at room temperature until the mixture was uniform. 0.27 g of potassium tert-butoxide was added to the uniform liquid, and the mixture was stirred at 70° C. for 23 hours to carry out a reaction.

Further, the compound represented by Formula (16) was synthesized by acting the intermediate compound represented by Formula (11) obtained by the first reaction of Example 1 with epibromohydrin.

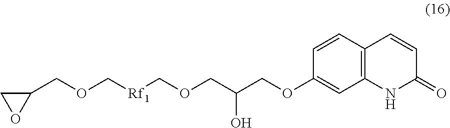

(In Rf₁ in Formula (16), h representing the average degree of polymerization represents 4.5 and i representing the average degree of polymerization represents 4.5.)

The reaction solution obtained after the reaction was cooled to room temperature, 5 g of a 10% hydrogen chloride/methanol solution (hydrogen chloride-methanol reagent (5-10%), manufactured by Tokyo Chemical Industry Co., Ltd.) was added thereto, and the mixture was stirred at room temperature for 2 hours. The reaction solution was transferred to a separatory funnel containing 100 mL of water, and was extracted three times with 100 mL of ethyl acetate. The organic layer was washed with water and dewatered with anhydrous sodium sulfate. The drying agent was separated by filtration, and the filtrate was concentrated, and the residues were purified by silica gel column chromatography, thereby obtaining 4.35 g of a compound (Q) (in two Rf₁'s in Formula (Q), h representing the average degree of polymerization represents 4.5 and i representing the average degree of polymerization represents 4.5).

The structure of the obtained compound (Q) was identified by performing ¹H-NMR measurement and ¹⁹F-NMR measurement based on the following results.

¹H-NMR (acetone-D₆): δ [ppm]=3.40-4.00 (23H), 4.10-4.20 (8H), 6.30-7.80 (6H)

¹⁹F-NMR (acetone-D₆): δ [ppm]=−55.5 to −51.5 (18F), −78.5 (4F), −80.5 (4F), −91.0 to −88.5 (36F)

### Example 18

A compound represented by Formula (R) was obtained by the following method.

4.52 g of a compound (R) (in two Rf₁'s in Formula (R), h representing the average degree of polymerization represents 4.5 and i representing the average degree of polymerization represents 4.5) was obtained by performing the same operation as in Example 17 except that a compound represented by Formula (13) was used instead of the compound represented by Formula (12), and a compound represented by Formula (7-2) was used instead of the compound represented by Formula (7-1) in Example 17.

The structure of the obtained compound (R) was identified by performing ¹H-NMR measurement and ¹⁹F-NMR measurement based on the following results.

¹H-NMR (acetone-D₆): δ [ppm]=3.40-4.00 (26H), 4.10-4.20 (8H), 5.20-6.00 (3H), 6.30-7.80 (5H)

¹⁹F-NMR (acetone-D₆): δ [ppm]=−55.5 to −51.5 (18F), −78.5 (4F), −80.5 (4F), −91.0 to −88.5 (36F)

### Example 19

A compound represented by Formula (S) was obtained by the following method.

4.52 g of a compound (S) (in two Rf₁'s in Formula (S), h representing the average degree of polymerization represents 4.5 and i representing the average degree of polymerization represents 4.5) was obtained by performing the same operation as in Example 15 except that a compound represented by Formula (7-7) was used instead of the compound represented by Formula (7-1) in Example 15.

The structure of the obtained compound (S) was identified by performing ¹H-NMR measurement and ¹⁹F-NMR measurement based on the following results.

¹H-NMR (acetone-D₆): δ [ppm]=3.40-4.00 (30H), 4.10-4.20 (8H), 6.30-7.80 (12H)

¹⁹F-NMR (acetone-D₆): δ [ppm]=−55.5 to −51.5 (18F), −78.5 (4F), −80.5 (4F), −91.0 to −88.5 (36F)

### Example 20

A compound represented by Formula (T) was obtained by the following method.

4.52 g of a compound (T) (in two Rf₁'s in Formula (T), h representing the average degree of polymerization represents 4.5 and i representing the average degree of polymerization represents 4.5) was obtained by performing the same operation as in Example 15 except that a compound represented by Formula (7-8) was used instead of the compound represented by Formula (7-1) in Example 15.

The structure of the obtained compound (T) was identified by performing ¹H-NMR measurement and ¹⁹F-NMR measurement based on the following results.

¹H-NMR (acetone-D₆): δ [ppm]=1.40-1.80 (4H), 3.40-4.00 (18H), 4.10-4.20 (8H), 6.30-7.80 (10H)

¹⁹F-NMR (acetone-D₆): δ [ppm]=−55.5 to −51.5 (18F), −78.5 (4F), −80.5 (4F), −91.0 to −88.5 (36F)

### Example 21

A compound represented by Formula (U) was obtained by the following method.

4.19 g of a compound (U) (in two Rf₁'s in Formula (U), h representing the average degree of polymerization represents 4.5 and i representing the average degree of polymerization represents 4.5) was obtained by performing the same operation as in Example 15 except that 2-(2-bromoethyl)oxirane was used instead of epibromohydrin in Example 15.

The structure of the obtained compound (U) was identified by performing ¹H-NMR measurement and ¹⁹F-NMR measurement based on the following results.

¹H-NMR (acetone-D₆): δ [ppm]=1.40-1.80 (2H), 3.40-4.00 (18H), 4.10-4.20 (8H), 6.30-7.80 (10H)

¹⁹F-NMR (acetone-D₆): δ [ppm]=−55.5 to −51.5 (18F), −78.5 (4F), −80.5 (4F), −91.0 to −88.5 (36F)

### Example 22

A compound represented by Formula (V) was obtained by the following method.

4.85 g of a compound (V) (in two Rf₂'s in Formula (V), j representing the average degree of polymerization represents 4.5) was obtained by performing the same operation as in Example 15 except that a compound represented by HOCH₂CF₂CF₂O(CF₂CF₂CF₂O)ⱼCF₂CF₂CH₂OH (j representing the average degree of polymerization in the formula represents 4.5) (number-average molecular weight: 1,000, molecular weight distribution: 1.1) was used instead of the compound represented by HOCH₂CF₂O(CF₂CF₂O)ₕ(CF₂O)ᵢCF₂CH₂OH in Example 15.

The structure of the obtained compound (V) was identified by performing ¹H-NMR measurement and ¹⁹F-NMR measurement based on the following results.

¹H-NMR (acetone-D₆): δ [ppm]=3.40-4.00 (18H), 4.10-4.20 (8H), 6.30-7.80 (12H)

¹⁹F-NMR (acetone-D₆): δ [ppm]=−84.0 to −83.0 (36F), −86.4 (8F), −124.3 (8F), −130.0 to −129.0 (18F)

### Example 23

A compound represented by Formula (W) was obtained by the following method.

A compound represented by HOCH₂CF₂O(CF₂CF₂O)ₕ(CF₂O)ᵢCF₂CH₂OH (h representing the average degree of polymerization in the formula represents 4.5, and i representing the average degree of polymerization in the formula represents 4.5) (number-average molecular weight: 1,000, molecular weight distribution: 1.1) reacted with epibromohydrin, thereby obtaining an intermediate compound represented by Formula (17).

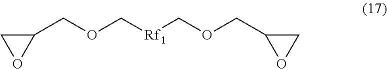

(In Rf₁ in Formula (17), h representing the average degree of polymerization represents 4.5, and i representing the average degree of polymerization represents 4.5.)

Subsequently, 4.75 g of a compound (W) (in three Rf₁'s in Formula (W), h representing the average degree of polymerization represents 4.5 and i representing the average degree of polymerization represents 4.5) was obtained by performing the same operation as in Example 15 except that the intermediate compound represented by Formula (17) was used instead of epibromohydrin in the reaction of the intermediate compound represented by Formula (11) with epibromohydrin in Example 15.

The structure of the obtained compound (W) was identified by performing ¹H-NMR measurement and ¹⁹F-NMR measurement based on the following results.

¹H-NMR (acetone-D₆): δ [ppm]=3.40-4.00 (24H), 4.10-4.20 (12H), 6.30-7.80 (12H)

¹⁹F-NMR (acetone-D₆): δ [ppm]=−55.5 to −51.5 (27F), −78.5 (6F), −80.5 (6F), −91.0 to −88.5 (54F)

### Example 24

A compound represented by Formula (X) was obtained by the following method.

4.55 g of a compound (X) (in three Rf₁'s in Formula (X), h representing the average degree of polymerization represents 4.5 and i representing the average degree of polymerization represents 4.5) was obtained by performing the same operation as in Example 16 except that the intermediate compound represented by Formula (17) was used instead of epibromohydrin in Example 16.

The structure of the obtained compound (X) was identified by performing ¹H-NMR measurement and ¹⁹F-NMR measurement based on the following results.

¹H-NMR (acetone-D₆): δ [ppm]=3.40-4.00 (24H), 4.10-4.20 (12H), 6.30-7.80 (10H)

¹⁹F-NMR (acetone-D₆): δ [ppm]=−55.5 to −51.5 (27F), −78.5 (6F), −80.5 (6F), −91.0 to −88.5 (54F)

### Example 25

A compound represented by Formula (Y) was obtained by the following method.

4.23 g of a compound (Y) (in three Rf₁'s in Formula (Y), h representing the average degree of polymerization represents 4.5 and i representing the average degree of polymerization represents 4.5) was obtained by performing the same operation as in Example 17 except that the intermediate compound represented by Formula (17) was used instead of epibromohydrin in the reaction of synthesizing the compound represented by Formula (16) in Example 17.

The structure of the obtained compound (Y) was identified by performing ¹H-NMR measurement and ¹⁹F-NMR measurement based on the following results.

¹H-NMR (acetone-D₆): δ [ppm]=3.40-4.00 (29H), 4.10-4.20 (12H), 6.30-7.80 (6H)

¹⁹F-NMR (acetone-D₆): δ [ppm]=−55.5 to −51.5 (27F), −78.5 (6F), −80.5 (6F), −91.0 to −88.5 (54F)

### Example 26

A compound represented by Formula (Z) was obtained by the following method.

4.84 g of a compound (Z) (in three Rf₁'s in Formula (Z), h representing the average degree of polymerization represents 4.5 and i representing the average degree of polymerization represents 4.5) was obtained by performing the same operation as in Example 19 except that the intermediate compound represented by Formula (17) was used instead of epibromohydrin in Example 19.

The structure of the obtained compound (Z) was identified by performing ¹H-NMR measurement and ¹⁹F-NMR measurement based on the following results.

¹H-NMR (acetone-D₆): δ [ppm]=3.40-4.00 (36H), 4.10-4.20 (12H), 6.30-7.80 (12H)

¹⁹F-NMR (acetone-D₆): δ [ppm]=−55.5 to −51.5 (27F), −78.5 (6F), −80.5 (6F), −91.0 to −88.5 (54F)

### Example 27

A compound represented by Formula (AA) was obtained by the following method.

A compound represented by HOCH₂CF₂O(CF₂CF₂O)ₕ(CF₂O)ᵢCF₂CH₂OH (h representing the average degree of polymerization in the formula represents 4.5, and i representing the average degree of polymerization in the formula represents 4.5) (number-average molecular weight: 700, molecular weight distribution: 1.1) reacted with 4-bromo-1-butene, thereby obtaining an intermediate compound represented by Formula (18) through the action of m-chloroperbenzoic acid.

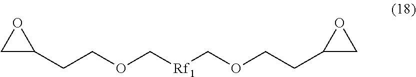

(In Rf₁ in Formula (18), h representing the average degree of polymerization represents 4.5, and i representing the average degree of polymerization represents 4.5.)

Subsequently, 4.62 g of a compound (AA) (in three Rf₁'s in Formula (AA), h representing the average degree of polymerization represents 4.5 and i representing the average degree of polymerization represents 4.5) was obtained by performing the same operation as in Example 15 except that the intermediate compound represented by Formula (18) was used instead of epibromohydrin in the reaction of the intermediate compound represented by Formula (11) with epibromohydrin in Example 15.

The structure of the obtained compound (AA) was identified by performing ¹H-NMR measurement and ¹⁹F-NMR measurement based on the following results.

¹H-NMR (acetone-D₆): δ [ppm]=1.40-1.80 (4H), 3.40-4.00 (24H), 4.10-4.20 (12H), 6.30-7.80 (12H)

¹⁹F-NMR (acetone-D₆): δ [ppm]=−55.5 to −51.5 (27F), −78.5 (6F), −80.5 (6F), −91.0 to −88.5 (54F)

### Example 28

A compound represented by Formula (AB) was obtained by the following method.

4.91 g of a compound (AB) (in three Rf₂'s in Formula (AB), j representing the average degree of polymerization represents 4.5) was obtained by performing the same operation as in Example 23 except that a compound represented by HOCH₂CF₂CF₂O(CF₂CF₂CF₂O)ⱼCF₂CF₂CH₂OH (j representing the average degree of polymerization in the formula represents 4.5) (number-average molecular weight: 1,000, molecular weight distribution: 1.1) was used instead of the compound represented by HOCH₂CF₂O(CF₂CF₂O)ₕ(CF₂O)ᵢCF₂CH₂OH in Example 23.

The structure of the obtained compound (AB) was identified by performing ¹H-NMR measurement and ¹⁹F-NMR measurement based on the following results.

¹H-NMR (acetone-D₆): δ [ppm]=3.40-4.00 (24H), 4.10-4.20 (12H), 6.30-7.80 (12H)

¹⁹F-NMR (acetone-D₆): δ [ppm]=−84.0 to −83.0 (54F), −86.4 (12F), −124.3 (12F), −130.0 to −129.0 (27F)

The structures of R¹ to R⁶ and the number of n in a case where the compounds (A) to (Z), (AA), and (AB) of Examples 1 to 28 obtained as described above were applied to Formula (1) are listed in Table 1.

TABLE 1

| Compound | R¹ | R² | R³ | R⁴ | R⁵ | R⁶ | n |
|---|---|---|---|---|---|---|---|
| (A) | (6-1) | (3-1) p1 = 1 | (5-1) | — | (3-3) p2 = 1 | —CH₂CH₂OH | 0 |
| (B) | (6-2) | (3-1) p1 = 1 | (5-1) | — | (3-3) p2 = 1 | —CH₂CH₂OH | 0 |
| (C) | (6-3) | (3-1) p1 = 1 | (5-1) | — | (3-3) p2 = 1 | —CH₂CH₂OH | 0 |
| (D) | (6-6) | (3-1) p1 = 1 | (5-1) | — | (3-3) p2 = 1 | —CH₂CH₂OH | 0 |
| (E) | (6-7) | (3-1) p1 = 1 | (5-1) | — | (3-3) p2 = 1 | —CH₂CH₂OH | 0 |
| (F) | (6-10) | (3-1) p1 = 1 | (5-1) | — | (3-3) p2 = 1 | —CH₂CH₂OH | 0 |
| (G) | (6-1) | (3-1) p1 = 1 | (5-1) | — | (3-3) p2 = 1 | (6-1) | 0 |
| (H) | (6-2) | (3-1) p1 = 1 | (5-1) | — | (3-3) p2 = 1 | (6-2) | 0 |
| (I) | (6-1) | (3-1) p1 = 1 | (5-1) | — | (3-3) p2 = 2 | Allyl group | 0 |
| (J) | (6-2) | (3-1) p1 = 1 | (5-1) | — | (3-3) p2 = 1 | Phenyl group | 0 |
| (K) | (6-1) | (3-1) p1 = 2 | (5-1) | — | (3-3) p2 = 1 | —CH₂CH₂OH | 0 |
| (L) | (6-2) | (3-2) q1 = 2 | (5-1) | — | (3-3) p2 = 1 | —CH₂CH₂OH | 0 |
| (M) | (6-1) | (3-1) p1 = 1 | (5-2) | — | (3-3) p2 = 1 | —CH₂CH₂OH | 0 |
| (N) | (6-2) | (3-1) p1 = 1 | (5-3) | — | (3-3) p2 = 1 | —CH₂CH₂OH | 0 |
| (O) | (6-1) | (3-1) p1 = 1 | (5-1) | (4-1) s1 = 1 | (3-3) p2 = 1 | (6-1) | 1 |
| (P) | (6-2) | (3-1) p1 = 1 | (5-1) | (4-1) s1 = 1 | (3-3) p2 = 1 | (6-2) | 1 |
| (Q) | (6-1) | (3-1) p1 = 1 | (5-1) | (4-1) s1 = 1 | (3-3) p2 = 1 | —CH₂CH₂OH | 1 |
| (R) | (6-2) | (3-1) p1 = 1 | (5-1) | (4-1) s1 = 1 | (3-3) p2 = 2 | Allyl group | 1 |
| (S) | (6-1) | (3-1) p1 = 2 | (5-1) | (4-1) s1 = 1 | (3-3) p2 = 2 | (6-1) | 1 |
| (T) | (6-2) | (3-2) q1 = 2 | (5-1) | (4-1) s1 = 1 | (3-4) r2 = 2 | (6-2) | 1 |
| (U) | (6-1) | (3-1) p1 = 1 | (5-1) | (4-3) u1 = 2 | (3-3) p2 = 1 | (6-1) | 1 |
| (V) | (6-1) | (3-1) p1 = 1 | (5-2) | (4-1) s1 = 1 | (3-3) p2 = 1 | (6-1) | 1 |
| (W) | (6-1) | (3-1) p1 = 1 | (5-1) | (4-1) s1 = 1 | (3-3) p2 = 1 | (6-1) | 2 |
| (X) | (6-2) | (3-1) p1 = 1 | (5-1) | (4-1) s1 = 1 | (3-3) p2 = 1 | (6-2) | 2 |
| (Y) | (6-1) | (3-1) p1 = 1 | (5-1) | (4-1) s1 = 1 | (3-3) p2 = 1 | —CH₂CH₂OH | 2 |
| (Z) | (6-1) | (3-1) p1 = 2 | (5-1) | (4-1) s1 = 1 | (3-3) p2 = 2 | (6-1) | 2 |
| (AA) | (6-1) | (3-1) p1 = 1 | (5-1) | R⁴ on R¹ side: | (3-3) p2 = 1 | (6-1) | 2 |
| | | | | (4-3) u1 = 2 | | | |
| | | | | R⁴ on R⁶ side: | | | |
| | | | | (4-2) t1 = 2 | | | |
| (AB) | (6-1) | (3-1) p1 = 1 | (5-2) | (4-1) s1 = 1 | (3-3) p2 = 1 | (6-1) | 2 |

### Comparative Example 1

A compound represented by Formula (AC) was synthesized by the method described in Patent Document 1.

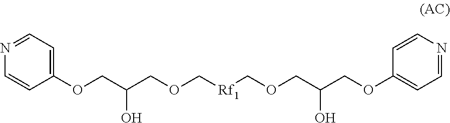

(In Rf₁ in Formula (AC), h representing the average degree of polymerization represents 4.5, and i represents 4.5.)

### Comparative Example 2

A compound represented by Formula (AD) was synthesized by the method described in Patent Document 2.

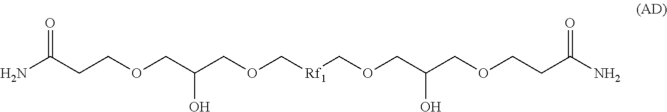

(In Rf₁ in Formula (AD), h representing the average degree of polymerization represents 4.5, and i represents 4.5.)

### Comparative Example 3

A compound represented by Formula (AE) was synthesized by the method described in Patent Document 3.

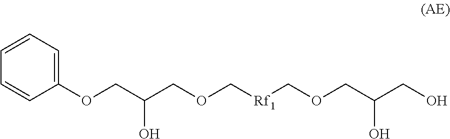

(In Rf₁ in Formula (AE), h representing the average degree of polymerization represents 4.5, and i represents 4.5.)

### Comparative Example 4

A compound represented by Formula (AF) was synthesized by the method described in Patent Document 4.

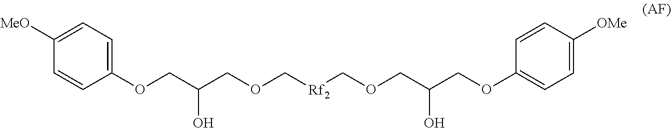

(In Rf₂ in Formula (AF), j representing the average degree of polymerization represents 7.0. Me represents a methyl group.)

### Comparative Example 5

A compound represented by Formula (AG) was synthesized by the method described in Patent Document 5.

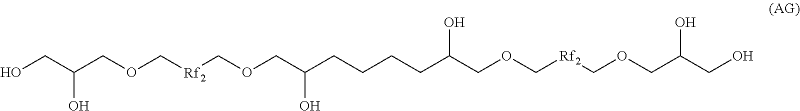

(In two Rf₂'s in Formula (AG), j representing the average degree of polymerization represents 7.0.)

The number-average molecular weights (Mn) of the compounds of Examples 1 to 28 and Comparative Examples 1 to 5, which were obtained as described above, were measured by the above-described method. The results thereof are listed in Tables 2 and 3.

Next, a lubricating layer forming solution was prepared using the compounds obtained in Examples 1 to 28 and Comparative Examples 1 to 5 by the following method. Next, a lubricating layer of a magnetic recording medium was formed by the following method using the obtained lubricating layer forming solution, thereby obtaining magnetic recording media of Examples 1 to 28 and Comparative Examples 1 to 5.

### “Solution for Forming Lubricating Layer”

The compounds obtained in Examples 1 to 28 and Comparative Examples 1 to 5 were each dissolved in VERTREL (registered trademark) XF (trade name, manufactured by DuPont Mitsui Fluorochemicals Co., Ltd.), which is a fluorine-based solvent, and diluted with VERTREL XF such that the film thickness in a case where the solution was applied onto the protective layer reached 9.0 Å to 9.5 Å, thereby preparing a lubricating layer forming solution.

### “Magnetic Recording Medium”

A magnetic recording medium in which an adhesive layer, a soft magnetic layer, a first underlayer, a second underlayer, a magnetic layer, and a protective layer were sequentially provided on a substrate having a diameter of 65 mm was prepared. The protective layer was formed of carbon.

The protective layer of the magnetic recording medium on which each layer up to the protective layer was formed was coated with each lubricating layer forming solution of Examples 1 to 28 and Comparative Examples 1 to 5 by a dipping method. Further, the dipping method was carried out under the conditions of an immersion speed of 10 mm/sec, an immersion time of 30 sec, and a pulling-up speed of 1.2 mm/sec.

Thereafter, the magnetic recording medium coated with the lubricating layer forming solution was placed in a constant temperature tank, and a thermal treatment was performed for 10 minutes to remove the solvent in the lubricating layer forming solution and to improve the adhesion between the protective layer and the lubricating layer so that a lubricating layer was formed on the protective layer, thereby obtaining a magnetic recording medium.

### (Measurement of Film Thickness)

The film thicknesses of the lubricating layers of the magnetic recording media of Examples 1 to 28 and Comparative Examples 1 to 5 obtained above were measured using FT-IR (trade name: Nicolet iS50, manufactured by Thermo Fisher Scientific). The results thereof are listed in Tables 2 and 3.

### (Chemical Substance Resistance Test)

Contamination of the magnetic recording medium due to an environmental substance generating a contaminant in a high-temperature environment was examined by the following method. A Si ion was used as the environmental substance, and a Si adsorption amount was measured as the amount of the contaminant which contaminated the magnetic recording medium generated by the environmental substance.

Specifically, the magnetic recording medium to be evaluated was held in a high-temperature environment of a temperature of 85° C. and a humidity of 0% for 240 hours in the presence of siloxane-based Si rubber. Next, the adsorption amount of Si present on the surface of the magnetic recording medium was analyzed and measured by secondary ion mass spectrometry (SIMS), and the degree of contamination with Si ions was evaluated as the Si adsorption amount. The Si adsorption amount was evaluated based on the following evaluation criteria using the numerical value in a case where the result of Comparative Example 5 was set to 1.00. The results thereof are listed in Tables 2 and 3.

### “Evaluation Criteria”

A: The Si adsorption amount was less than 0.70.
B: The Si adsorption amount was 0.70 or greater and less than 0.90.
C: The Si adsorption amount was 0.90 or greater and less than 1.10.
D: The Si adsorption amount was 1.10 or greater.

### (Pickup Suppression Test)

The magnetic recording medium and the magnetic head were mounted on a spin stand, and the magnetic head was floated from a fixed point for 10 minutes at room temperature under reduced pressure (about 250 torr). Thereafter, the surface (surface of the lubricating layer) of the magnetic head facing the magnetic recording medium was analyzed using an analyzer of electron spectroscopy for chemical analysis (ESCA). Further, the adhesion amount of the lubricant to the magnetic head was evaluated based on the intensity (signal intensity (a.u.)) of the fluorine-derived peak measured by ESCA according to the following criteria. The results thereof are listed in Tables 2 and 3.

### “Evaluation Criteria”

A: The ESCA signal intensity was 500 or less, which was extremely small (the adhesion amount of the lubricant to the head was extremely small).
B: The ESCA signal intensity was 501 or greater and 800 or less, which was small (the adhesion amount of the lubricant to the head was small).
C: The ESCA signal intensity was 801 or greater and 1000 or less, which was slightly large (the adhesion amount of the lubricant to the head was slightly large).
D: The ESCA signal intensity was 1001 or greater, which was large (the adhesion amount of the lubricant to the head was large).

TABLE 2

| | | Number | Film | Chemical | Pickup |
|---|---|---|---|---|---|
| | | average | thick- | substance | suppres- |
| | | molecular | ness | resistance | sion |
| | Compound | weight | (Å) | test | test |
| Example 1 | (A) | 1342 | 9.5 | B | A |
| Example 2 | (B) | 1334 | 9.5 | B | A |
| Example 3 | (C) | 1321 | 9.5 | B | A |
| Example 4 | (D) | 1384 | 9.4 | B | A |
| Example 5 | (E) | 1328 | 9.5 | B | A |
| Example 6 | (F) | 1401 | 9.3 | B | A |
| Example 7 | (G) | 1456 | 9.1 | A | B |
| Example 8 | (H) | 1424 | 9.1 | A | B |
| Example 9 | (I) | 1427 | 9.3 | A | B |
| Example 10 | (J) | 1375 | 9.5 | A | B |
| Example 11 | (K) | 1402 | 9.2 | B | A |
| Example 12 | (L) | 1362 | 9.3 | A | B |
| Example 13 | (M) | 1351 | 9.3 | B | A |
| Example 14 | (N) | 1367 | 9.2 | B | A |
| Example 15 | (O) | 2552 | 9.2 | A | A |
| Example 16 | (P) | 2531 | 9.1 | A | A |
| Example 17 | (Q) | 2423 | 9.0 | B | A |
| Example 18 | (R) | 2505 | 9.2 | A | B |
| Example 19 | (S) | 2631 | 9.0 | B | A |
| Example 20 | (T) | 2599 | 9.0 | A | B |
| Example 21 | (U) | 2546 | 9.0 | A | B |
| Example 22 | (V) | 2532 | 9.0 | A | A |
| Example 23 | (W) | 3543 | 9.2 | A | A |
| Example 24 | (X) | 3536 | 9.5 | A | A |
| Example 25 | (Y) | 3442 | 9.3 | B | A |
| Example 26 | (Z) | 3704 | 9.1 | B | A |
| Example 27 | (AA) | 3571 | 9.4 | A | B |
| Example 28 | (AB) | 3532 | 9.3 | A | A |

TABLE 3

| | | Number | Film | Chemical | Pickup |
|---|---|---|---|---|---|
| | | average | thick- | substance | suppres- |
| | | molecular | ness | resistance | sion |
| | Compound | weight | (Å) | test | test |
| Comparative | (AC) | 1315 | 9.4 | B | D |
| Example 1 | | | | | |
| Comparative | (AD) | 1283 | 9.5 | B | C |
| Example 2 | | | | | |
| Comparative | (AE) | 1235 | 9.4 | D | C |
| Example 3 | | | | | |
| Comparative | (AF) | 1358 | 9.4 | C | D |
| Example 4 | | | | | |
| Comparative | (AG) | 2273 | 9.0 | C | C |
| Example 5 | | | | | |

As shown in Table 2, in all the magnetic recording media of Examples 1 to 28, the evaluation was A or B in all the evaluation items. Therefore, it was confirmed that the lubricating layer of the magnetic recording medium of Examples 1 to 28 had satisfactory chemical substance resistance and excellent pickup suppression.

In particular, in Examples 7, 8, 15, 16, 20 to 24, and 27 and 28, the chemical substance resistance was satisfactory as compared with Examples 19 and 26. The reason for this was considered to be that, in Examples 7, 8, 15, 16, 20 to 24, and 27 and 28, since the compound in which both R¹ and R⁶ represent a condensed aromatic group represented by Formula (2) and the numbers of hydroxy groups in R² and R⁵ were each 1, or the total number of hydroxy groups in R² and R⁵ was appropriate, was used, the number of polar groups not involved in the bonding to the active point on the protective layer was small.

In Examples 9, 10, and 18 in which the compound in which only one of R¹ or R⁶ represents a condensed aromatic ring represented by Formula (2) and the other represents an organic group having a double bond was used and in Example 12 in which a compound in which R² is represented by Formula (3-2) was used, the chemical substance resistance was satisfactory. The reason for this was considered to be that the interaction between the polar group and Si, which were not involved in the bonding to the active point on the protective layer was weakened due to a decrease in the polarity of the entire molecule.

In Examples 15, 16, 19, 22, 23, 24, 26, and 28, the evaluation of the pickup suppression test was satisfactory as compared with Examples 20, 21, and 27. The reason for this was considered to be that, in Examples 15, 16, 19, 22, 23, 24, 26, and 28, the compound in which both R¹ and R⁶ in Formula (1) represent a condensed aromatic group represented by Formula (2), the total number of hydroxy groups in R², R⁴, and R⁵ was 3 or more, and at least one of R², R⁴, and R⁵ had a flexible glycerin structure (—OCH₂CH(OH)CH₂O—) was used, and thus the interaction with the protective layer was strengthened.

In addition, in Examples 15, 16, 19, 22, 23, 24, 26, and 28, the evaluation of the pickup suppression test was satisfactory as compared with Examples 7 and 8 in which the compound in which both R¹ and R⁶ in Formula (1) represent a condensed aromatic group represented by Formula (2) and the total number of hydroxy groups included in R² and R⁵ was 2 was used. The reason for this was considered to be that in Examples 15, 16, 19, 22, 23, 24, 26, and 28, the number of hydroxy groups contained in the molecule was appropriate, and thus a sufficient adsorption force to the protective layer was obtained.

In Examples 1, 2, 3, 4, 5, 6, 11, 13, 14, 17, and 25 in which the compound in which only one of R¹ or R⁶ represents a condensed aromatic group represented by Formula (2) and the other represents an organic group having a primary hydroxy group was used, the evaluation of the pickup suppression test was satisfactory. The reason for this was considered to be that since the primary hydroxy group contained in the above-described compound had a strong adsorption force on the protective layer, the lubricating layer could sufficiently adhere to the protective layer.

In addition, in Examples 1, 2, 3, 4, 5, 6, 11, 13, 14, 17, and 25, the evaluation of the pickup suppression test was satisfactory as compared with Example 12 in which the compound in which one of R¹ or R⁶ in Formula (1) represents a condensed aromatic group represented by Formula (2) and the other represents an organic group having a primary hydroxy group, and R² has a structure obtained by carbon-increasing the glycerin structure (—OCH₂CH(OH)CH₂O—) was used. The reason for this was considered to be that, in Examples 1, 2, 3, 4, 5, 6, 11, 13, 14, 17, and 25, the flexible glycerin structure in R², R⁴, and R⁵ made the interaction with the protective layer strong.

The compounds of Comparative Examples 1 and 2 had pyridine or amide through a glycerin structure (—OCH₂CH(OH)CH₂O—) at both ends of the perfluoropolyether chain. In Comparative Examples 1 and 2, the evaluation results of the chemical substance resistance test were both B, but the evaluation results of the pickup suppression test were D and C. The reason for this was considered to be that in Comparative Examples 1 and 2, the adsorption force of pyridine or amide at the end with respect to the protective layer was insufficient, and thus the lubricating layer did not sufficiently adhere to the protective layer.

In the compounds of Comparative Examples 3 and 4, an end group having a benzene ring was disposed at one or both ends of the perfluoropolyether chain through the glycerin structure (—OCH₂CH(OH)CH₂O—). In Comparative Examples 3 and 4, the evaluation results of the chemical substance resistance test were D and C, and the evaluation results of the pickup suppression test were C and D. The reason why satisfactory results were not obtained in the evaluation of the chemical substance resistance was considered to be that since the benzene ring of the end group had almost no polarity, the interaction between the end group and the protective layer was hardly obtained, and the thus the lubricating layer was not sufficiently wet and spread on the protective layer. In addition, it was considered that the effect of suppressing the pickup was not sufficiently obtained because the lubricating layer did not sufficiently adhere to the protective layer.

In the compound of Comparative Example 5, an alkyl chain having 8 carbon atoms and two hydroxy groups was disposed in the center of the chain structure, a perfluoropolyether chain and an end group having two hydroxy groups disposed at the most ends of the chain structure were bonded to both sides of the alkyl chain in this order. In Comparative Example 5, the evaluation result of the chemical substance resistance test and the evaluation result of the pickup suppression test were both C. This was considered to be caused by the fact that, since there were many hydroxy groups that were not involved in the bonding to the active point on the protective layer, Si was likely to be mixed into the lubricating layer, and the adsorption force on the protective layer was insufficient.

### INDUSTRIAL APPLICABILITY

By using the lubricant for a magnetic recording medium containing the fluorine-containing ether compound of the present invention, a lubricating layer which has excellent adhesion even in a case where the thickness thereof is small, has satisfactory chemical substance resistance, and suppresses the pickup can be formed.

### REFERENCE SIGNS LIST

10: magnetic recording medium
11: substrate
12: adhesive layer
13: soft magnetic layer
14: first underlayer
15: second underlayer
16: magnetic layer
17: protective layer
18: lubricating layer

1: A fluorine-containing ether compound which is represented by Formula (1),
R¹—R²—CH₂—R³[—CH₂—R⁴—CH₂—R³]ₙ—CH₂—R⁵—R⁶ (1)
(in Formula (1), n represents an integer of 0 to 2; R³ represents a perfluoropolyether chain; (n+1) pieces of R³'s may be partially or entirely the same as or different from each other; R⁴ represents a divalent linking group having one or more polar groups; two R⁴'s may be the same as or different from each other in a case where n represents 2; Each R² and R⁵ represents a divalent linking group having one or more polar groups; R² and R⁵ may be the same as or different from each other; R² has an oxygen atom at an end bonded to R¹; R⁵ has an oxygen atom at an end bonded to R⁶; Each R¹ and R⁶ represents an organic group having 1 to 50 carbon atoms, which is bonded to an oxygen atom included in R² or R⁵, or a hydrogen atom; R¹ and R⁶ may be the same as or different from each other; and at least one of R¹ or R⁶ has a structure represented by Formula (2)), 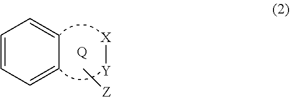
(in Formula (2), one of X and Y represents a carbonyl group (C═O), and the other represents —NH—, —NR⁷— (R⁷ represents a hydrocarbon group having 1 to 8 carbon atoms), or —O—; Q represents that a carbon atom of a linking group indicated by a dashed arc forms a 5- to 8-membered heterocyclic ring structure together with X and Y; Z represents 0 to 5 substituents; Z's each independently represent an organic group having 1 to 8 carbon atoms; Z may be bonded to any of carbon atoms constituting two rings in Formula (2) (where a carbonyl carbon atom of X or Y is excluded); in a case where two or more Z's are present, two Z's may be bonded to each other to form a ring structure; and any of the carbon atoms constituting the two rings in Formula (2) (where a carbonyl carbon atom of X or Y is excluded) is bonded to the oxygen atom at the end of R² or R⁵).

2: The fluorine-containing ether compound according to claim 1,
wherein Formula (2) is a structure represented by Formula (2-1), 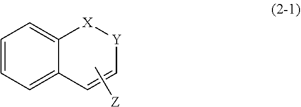
(in Formula (2-1), one of X and Y represents a carbonyl group (C═O), and the other represents —NH—, —NR⁷— (R⁷ represents a hydrocarbon group having 1 to 8 carbon atoms), or —O—; Z represents 0 to 5 substituents; Z's each independently represent an organic group having 1 to 8 carbon atoms; Z may be bonded to any of carbon atoms constituting two rings in Formula (2-1) (where a carbonyl carbon atom of X or Y is excluded); in a case where two or more Z's are present, two Z's may be bonded to each other to form a ring structure; and any of the carbon atoms constituting the two rings in Formula (2-1) (where a carbonyl carbon atom of X or Y is excluded) is bonded to the oxygen atom at the end of R² or R⁵.)

3: The fluorine-containing ether compound according to claim 1,
wherein R¹ and R⁶ in Formula (1) are the same as each other.

4: The fluorine-containing ether compound according to claim 1,
wherein one of R¹ and R⁶ in Formula (1) represents the structure represented by Formula (2) and the other represents an organic group, an alkyl group, or a hydrogen atom, the organic group having at least one double bond or triple bond and being other than the structure represented by Formula (2), the alkyl group having 1 to 8 carbon atoms, which may have a substituent.

5: The fluorine-containing ether compound according to claim 1,
wherein the organic group as Z in Formula (2) is selected from the group consisting of an alkyl group, an alkenyl group, an alkynyl group, an aryl group, an alkyl group substituted with an aryl group, and an alkoxy group.

6: The fluorine-containing ether compound according to claim 1,
wherein all the polar groups in R², R⁴, and R⁵ in Formula (1) are hydroxy groups.

7: The fluorine-containing ether compound according to claim 1,
wherein some or all of the polar groups in R², R⁴, and R⁵ in Formula (1) are hydroxy groups, and
a total number of hydroxy groups included in R², hydroxy groups included in R⁴, and hydroxy groups included in R⁵ is in a range of 2 to 8.

8: The fluorine-containing ether compound according to claim 1,
wherein R² and R⁵ in Formula (1) each independently represent a linking group represented by Formula (3), 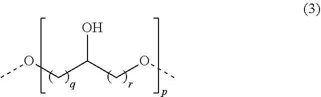
(in Formula (3), p represents an integer of 1 to 3; q represents an integer of 1 to 5; r represents an integer of 1 to 5; in a case where p represents 2 or greater, combinations of q and r in each structural unit may be the same as or different from each other; in Formula (3), an oxygen atom at an end on a left side is bonded to a group disposed on an R¹ side of R² or R⁵ in Formula (1); in Formula (3), an oxygen atom at an end on a right side is bonded to a group disposed on an R⁶ side of R² or R⁵ in Formula (1)).

9: The fluorine-containing ether compound according to claim 1,
wherein R² in Formula (1) represents a linking group represented by Formula (3-1) or (3-2), and R⁵ in Formula (1) represents a linking group represented by Formula (3-3) or (3-4), 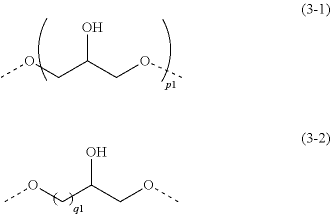
(p1 in Formula (3-1) represents an integer of 1 to 3; q1 in Formula (3-2) represents an integer of 2 to 4; and in Formulae (3-1) and (3-2), an oxygen atom at an end on a left side is bonded to R¹ in Formula (1)), 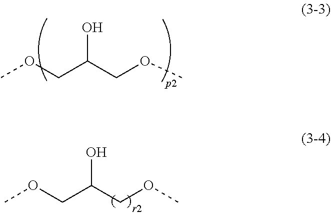
(p2 in Formula (3-3) represents an integer of 1 to 3; r2 in Formula (3-4) represents an integer of 2 to 4; and in Formulae (3-3) and (3-4), an oxygen atom at an end on a right side is bonded to R⁶ in Formula (1)).

10: The fluorine-containing ether compound according to claim 1,
wherein in Formula (1), the (n+1) pieces of R³'s are the same as each other, and R¹—R²— and R⁶—R⁵— are the same as each other.

11: The fluorine-containing ether compound according to claim 1,
wherein n pieces of R⁴'s in Formula (1) each independently represent a linking group represented by Formula (4), 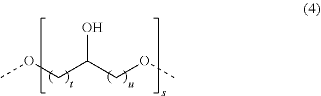
(in Formula (4), s represents an integer of 1 to 3; t represents an integer of 1 to 5; u represents an integer of 1 to 5; in a case where s represents 2 or greater, combinations of t and u in each structural unit may be the same as or different from each other; in Formula (4), an oxygen atom at an end on a left side is bonded to a group disposed on an R¹ side of R⁴ in Formula (1); and in Formula (4), an oxygen atom at an end on a right side is bonded to a group disposed on an R⁶ side of R⁴ in Formula (1)).

12: The fluorine-containing ether compound according to claim 1,
wherein n pieces of R⁴'s in Formula (1) each independently represent a linking group represented by any of Formulae (4-1) to (4-3), 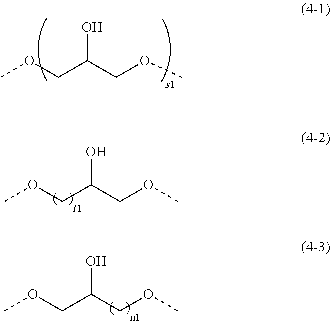
(s1 in Formula (4-1) represents an integer of 1 to 3),
(t1 in Formula (4-2) represents an integer of 2 to 4; and in Formula (4-2), an oxygen atom at an end on a left side is bonded to a methylene group adjacent to an R¹ side of R⁴ in Formula (1)),
(u1 in Formula (4-3) represents an integer of 2 to 4; and in Formula (4-3), an oxygen atom at an end on a right side is bonded to a methylene group adjacent to an R⁶ side of R⁴).

13: The fluorine-containing ether compound according to claim 1,
wherein the (n+1) pieces of R³'s in Formula (1) each independently represent a perfluoropolyether chain represented by Formula (5),
−(CF₂)_{w1}−O—(CF₂O)_{w2}−(CF₂CF₂O)_{w3}−(CF₂CF₂CF₂O)_{w4}−(CF₂CF₂CF₂CF₂O)_{w5}−(CF₂)_{w6}− (5)
(in Formula (5), w2, w3, w4, and w5 represent an average degree of polymerization, and each independently represent 0 to 20, where all of w2, w3, w4, and w5 do not represent 0 at the same time; w1 and w6 represent an average value representing the number of CF₂'s, and each independently represent 1 to 3; and an arrangement order of (CF₂O), (CF₂CF₂O), (CF₂CF₂CF₂O), and (CF₂CF₂CF₂CF₂O), which are the repeating units in Formula (5), is not particularly limited).

14: The fluorine-containing ether compound according to claim 1,
wherein the (n+1) pieces of R³'s in Formula (1) each independently represent any one selected from the group consisting of perfluoropolyether chains each represented by any of Formulae (5-1) to (5-4),
—CF₂—(OCF₂CF₂)ₕ—(OCF₂)ᵢ—OCF₂— (5-1)
(in Formula (5-1), h and i represent an average degree of polymerization, where h represents 1 to 20 and i represents 0 to 20),
—CF₂CF₂—(OCF₂CF₂CF₂)ⱼ—OCF₂CF₂— (5-2)
(in Formula (5-2), j represents an average degree of polymerization, and represents 1 to 15),
—CF₂CF₂CF₂—(OCF₂CF₂CF₂CF₂)ₖ—OCF₂CF₂CF₂— (5-3)
(in Formula (5-3), k represents an average degree of polymerization, and represents 1 to 10),
—(CF₂)_{w7}—O—(CF₂CF₂CF₂O)_{w8}—(CF₂CF₂O)_{w9}—(CF₂)_{w10}— (5-4)
(in Formula (5-4), w8 and w9 represent an average degree of polymerization, and each independently represents 1 to 20; and w7 and w10 represent an average value representing the number of CF₂'s, and each independently represent 1 to 2).

15: The fluorine-containing ether compound according to claim 1,
wherein the fluorine-containing ether compound has a number-average molecular weight of 500 to 10000.

16: A lubricant for a magnetic recording medium, comprising:
the fluorine-containing ether compound according to claim 1.

17: A magnetic recording medium, which is provided with at least a magnetic layer, a protective layer, and a lubricating layer in this order on a substrate,
wherein the lubricating layer contains the fluorine-containing ether compound according to claim 1.

18: The magnetic recording medium according to claim 17,
wherein the lubricating layer has an average film thickness of 0.5 nm to 2.0 nm.
